SEQUENCE LISTING (154 sequences)

SEQ ID NO: 1 - DNA, 16 bp, Homo sapiens
ggtagcgtggccgagc

SEQ ID NO: 2 - DNA, 15 bp, Homo sapiens
ggccggttagctcag

SEQ ID NO: 3 - DNA, 18 bp, Homo sapiens
gggccagtggcgcaatgg

SEQ ID NO: 4 - DNA, 23 bp, Homo sapiens
tcccacatggtctagcggttagg

SEQ ID NO: 5 - DNA, 13 bp, Homo sapiens
gggggtatagctc

SEQ ID NO: 6 - DNA, 30 bp, Homo sapiens
gggggtgtagctcagtggtagagcgcgtgc

SEQ ID NO: 7 - DNA, 14 bp, Homo sapiens
gacccagtggccta

SEQ ID NO: 8 - DNA, 18 bp, Homo sapiens
tcctcgttagtatagtgg

SEQ ID NO: 9 - DNA, 23 bp, Homo sapiens
tcctcgttagtatagtggtgagt

SEQ ID NO: 10 - DNA, 32 bp, Homo sapiens
gggggtatagctcagtggtagagcatttgact

SEQ ID NO: 11 - DNA, 30 bp, Homo sapiens
ggggtatagctcaggggagagcatttgact

SEQ ID NO: 12 - DNA, 14 bp, Homo sapiens
ggttccatggtgta

SEQ ID NO: 13 - DNA, 23 bp, Homo sapiens
tccctggtggtctagtggttagg

SEQ ID NO: 14 - DNA, 23 bp, Homo sapiens
tcccatatggtctagcggttagg

SEQ ID NO: 15 - DNA, 23 bp, Homo sapiens
tccctgtggtctagtggttagga

SEQ ID NO: 16 - DNA, 30 bp, Homo sapiens
gcgccgctggtgtagtggtatcatgcaaga

SEQ ID NO: 17 - DNA, 22 bp, Homo sapiens
gcatgggtggttcagtggtaga

SEQ ID NO: 18 - DNA, 22 bp, Homo sapiens
gcattggtggttcagtggtaga

SEQ ID NO: 19 - DNA, 23 bp, Homo sapiens
gcgttggtggtatagtggtgagc

SEQ ID NO: 20 - DNA, 23 bp, Homo sapiens
gtcaggatggccgagcggtctaa

SEQ ID NO: 21 - DNA, 30 bp, Homo sapiens
gttaagatggcagagcccggtaatcgcata

SEQ ID NO: 22 - DNA, 19 bp, Homo sapiens
gttaagatggcagagcccg

SEQ ID NO: 23 - DNA, 31 bp, Homo sapiens
gcccggctagctcagtcggtagagcatgaga

SEQ ID NO: 24 - DNA, 32 bp, Homo sapiens
gcccggctagctcagtcggtagagcatgggac

SEQ ID NO: 25 - DNA, 31 bp, Homo sapiens
gcccggatagctcagtcggtagagcatcaga

SEQ ID NO: 26 - DNA, 30 bp, Homo sapiens
ggctcgttggtctaggggtatgattctcgc

SEQ ID NO: 27 - DNA, 17 bp, Homo sapiens
ggctcgttggtctaggg

SEQ ID NO: 28 - DNA, 15 bp, Homo sapiens
ggctcgttggtctag

SEQ ID NO: 29 - DNA, 23 bp, Homo sapiens
gtagtcgtggccgagtggttaag

SEQ ID NO: 30 - DNA, 15 bp, Homo sapiens
ccttcgatagctcag

SEQ ID NO: 31 - DNA, 31 bp, Homo sapiens
gtttccgtagtgtagtggtcatcacgttcgc

SEQ ID NO: 32 - DNA, 31 bp, Homo sapiens
gcttctgtagtgtagtggttatcacgttcgc

SEQ ID NO: 33 - DNA, 31 bp, Homo sapiens
gtttccgtagtgtagtggttatcacgttcgc

SEQ ID NO: 34 - DNA, 31 bp, Homo sapiens
ggttccatagtgtagtggttatcacgtctgc

SEQ ID NO: 35 - DNA, 18 bp, Homo sapiens
atcccaccgctgccacca

SEQ ID NO: 36 - DNA, 18 bp, Homo sapiens
tccccggcacctccacca

SEQ ID NO: 37 - DNA, 18 bp, Homo sapiens
tccccggcatctccacca

SEQ ID NO: 38 - DNA, 22 bp, Homo sapiens
tcgattcccggcccatgcacca

SEQ ID NO: 39 - DNA, 22 bp, Homo sapiens
tcgattcccggccaacgcacca

SEQ ID NO: 40 - DNA, 18 bp, Homo sapiens
accccactcctggtacca

SEQ ID NO: 41 - DNA, 17 bp, Homo sapiens
accgggcggaaacacca

SEQ ID NO: 42 - DNA, 17 bp, Homo sapiens
cccacccagggacgcca

SEQ ID NO: 43 - DNA, 17 bp, Homo sapiens
tccctgttcgggcgcca

SEQ ID NO: 44 - DNA, 18 bp, Homo sapiens
atcccactcctgacacca

SEQ ID NO: 45 - DNA, 22 bp, Homo sapiens
tcgattcccggccaatgcacca

SEQ ID NO: 46 - DNA, 18 bp, Homo sapiens
atcccggacgagccccca

SEQ ID NO: 47 - DNA, 18 bp, Homo sapiens
atcccaccactgccacca

SEQ ID NO: 48 - DNA, 18 bp, Homo sapiens
atcccacttctgacacca

SEQ ID NO: 49 - DNA, 17 bp, Homo sapiens
cccgggtttcggcacca

SEQ ID NO: 50 - DNA, 17 bp, Homo sapiens
tccgggtgccccctcca

SEQ ID NO: 51 - DNA, 17 bp, Homo sapiens
tccgagtcacggcacca

SEQ ID NO: 52 - DNA, 18 bp, Homo sapiens
tccccgtacgggccacca

SEQ ID NO: 53 - DNA, 22 bp, Homo sapiens
tcgattccccgacggggagcca

SEQ ID NO: 54 - DNA, 17 bp, Homo sapiens
tccggctcgaaggacca

SEQ ID NO: 55 - DNA, 17 bp, Homo sapiens
tctcgctggggcctcca

SEQ ID NO: 56 - DNA, 22 bp, Homo sapiens
tcgagccccagtggaaccacca

SEQ ID NO: 57 - DNA, 17 bp, Homo sapiens
tctcggtggaacctcca

SEQ ID NO: 58 - DNA, 17 bp, Homo sapiens
tctcggtgggacctcca

SEQ ID NO: 59 - DNA, 18 bp, Homo sapiens
tccccagtacctccacca

SEQ ID NO: 60 - DNA, 25 bp, Homo sapiens
ggttcgattccccgacggggagcca

SEQ ID NO: 61 - DNA, 22 bp, Homo sapiens
tcgattcccggtcagggaacca

SEQ ID NO: 62 - DNA, 22 bp, Homo sapiens
tcgtttcccggtcagggaacca

SEQ ID NO: 63 - DNA, 22 bp, Homo sapiens
tcgactcccggtgtgggaacca

SEQ ID NO: 64 - DNA, 22 bp, Homo sapiens
tcgagccccacgttgggcgcca

SEQ ID NO: 65 - DNA, 22 bp, Homo sapiens
tcgagcctcagagagggcacca

SEQ ID NO: 66 - DNA, 18 bp, Homo sapiens
atcctgccgactacgcca

SEQ ID NO: 67 - DNA, 17 bp, Homo sapiens
tccggctcggaggacca

SEQ ID NO: 68 - DNA, 20 bp, Homo sapiens
gaagcgggtgctcttatttt

SEQ ID NO: 69 - DNA, 26 bp, Homo sapiens
gctaaggaagtcctgtgctcagtttt

SEQ ID NO: 70 - DNA, 18 bp, Homo sapiens
gtgtgtagctgcactttt

SEQ ID NO: 71 - DNA, 17 bp, Homo sapiens
atgtggtggcttacttt

SEQ ID NO: 72 - DNA, 19 bp, Homo sapiens
gtgtaagcagggtcgtttt

SEQ ID NO: 73 - DNA, 17 bp, Homo sapiens
ttcaaaggtgaacgttt

SEQ ID NO: 74 - DNA, 18 bp, Homo sapiens
tagggtgtgcgtgttttt

SEQ ID NO: 75 - DNA, 19 bp, Homo sapiens
tggtgtggtctgttgtttt

SEQ ID NO: 76 - DNA, 17 bp, Homo sapiens
gcacgaaaatgtgtttt

SEQ ID NO: 77 - DNA, 18 bp, Homo sapiens
ggcgatcacgtagatttt

SEQ ID NO: 78 - DNA, 23 bp, Homo sapiens
tgtgctccggagttacctcgttt

SEQ ID NO: 79 - DNA, 17 bp, Homo sapiens
gagagcgctcggttttt

SEQ ID NO: 80 - DNA, 15 bp, Homo sapiens
gcgggcggacctttt

SEQ ID NO: 81 - DNA, 15 bp, Homo sapiens
gcaactggtcgtttt

SEQ ID NO: 82 - DNA, 28 bp, Homo sapiens
gagggttctcaccttctctctccgattt

SEQ ID NO: 83 - DNA, 17 bp, Homo sapiens
ttccgtgggtttgtttt

SEQ ID NO: 84 - DNA, 16 bp, Homo sapiens
atggccgcatatattt

SEQ ID NO: 85 - DNA, 15 bp, Homo sapiens
gaggcttaaactttt

SEQ ID NO: 86 - DNA, 17 bp, Homo sapiens
ataggtattaaggtttt

SEQ ID NO: 87 - DNA, 15 bp, Homo sapiens
ggaatgtcagctttt

SEQ ID NO: 88 - DNA, 16 bp, Homo sapiens
acaagtgcggtttttt

SEQ ID NO: 89 - DNA, 15 bp, Homo sapiens
aagaggagttgtttt

SEQ ID NO: 90 - DNA, 36 bp, Homo sapiens
gtggggtgcctcacagcttcgctgcgtgagcatttt

SEQ ID NO: 91 - DNA, 23 bp, Homo sapiens
aagggaggttatgattaactttt

SEQ ID NO: 92 - DNA, 36 bp, Homo sapiens
gatatccaaccttcggctatagggtggagacttttt

SEQ ID NO: 93 - DNA, 15 bp, Homo sapiens
gggttgctgtctttt

SEQ ID NO: 94 - DNA, 20 bp, Homo sapiens
acctcagaaggtctcacttt

SEQ ID NO: 95 - DNA, 16 bp, Homo sapiens
aggtgaaagttccttt

SEQ ID NO: 96 - DNA, 31 bp, Homo sapiens
tcgagaggggctgtgctcgcaaggtttcttt

SEQ ID NO: 97 - DNA, 15 bp, Homo sapiens
gtgggtggctttttt

SEQ ID NO: 98 - DNA, 15 bp, Homo sapiens
tgcggtaccactttt

SEQ ID NO: 99 - DNA, 28 bp, Homo sapiens
aaccgagcgtccaagctctttccatttt

SEQ ID NO: 100 - DNA, 12 bp, Homo sapiens
aggttgggtttt

SEQ ID NO: 101 - DNA, 12 bp, Pan troglodytes
aggttgggtttt

SEQ ID NO: 102 - DNA, 16 bp, Macaca mulatta
ggggtggttgtgtttt

SEQ ID NO: 103 - DNA, 11 bp, Mus musculus
agttaggtttt

SEQ ID NO: 104 - DNA, 16 bp, Pongo pygmaeus
aacgaagaattgtttt

SEQ ID NO: 105 - DNA, 15 bp, Macaca mulatta
gcgggccgacctttt

SEQ ID NO: 106 - DNA, 15 bp, Pongo pygmaeus
gcgggccgacctttt

SEQ ID NO: 107 - DNA, 15 bp, Mus musculus
gcgtgcccacgtttt

SEQ ID NO: 108 - DNA, 15 bp, Pan troglodytes
tgcgatgttactttt

SEQ ID NO: 109 - DNA, 17 bp, homo sapiens
cctgttggcttactttt

SEQ ID NO: 110 - DNA, 17 bp, Pan troglodytes
cctgttggcttcctttt

SEQ ID NO: 111 - DNA, 17 bp, macaca mulatta
cctgtcggcttactttt

SEQ ID NO: 112 - DNA, 13 bp, mus musculus
taaggttactttt

SEQ ID NO: 113 - DNA, 15 bp, pongo pygmaeus
tctggaattaatttc

SEQ ID NO: 114 - DNA, 16 bp, homo sapiens
cttcgtctgtaatttt

SEQ ID NO: 115 - DNA, 16 bp, Pan troglodytes
cttcgtctgtaatttt

SEQ ID NO: 116 - DNA, 17 bp, mus musculus
cttcgtgcactactttt

SEQ ID NO: 117 - DNA, 16 bp, macaca mulatta
cttcgtgtaccatttt

SEQ ID NO: 118 - DNA, 16 bp, Pongo pygmaeus
cttcgtgtatcatttt

SEQ ID NO: 119 - DNA, 13 bp, homo sapiens
ggcttaaactttt

SEQ ID NO: 120 - DNA, 16 bp, Pongo pygmaeus
aagacctaagcctttt

SEQ ID NO: 121 - DNA, 14 bp, Macaca mulatta
gagacttcagtttt

SEQ ID NO: 122 - DNA, 14 bp, Pan troglodytes
gaggcttaagtttt

SEQ ID NO: 123 - DNA, 16 bp, mus musculus
aagatggctaaatttt

SEQ ID NO: 124 - DNA, 17 bp, Pan troglodytes
aagaaaggctgaatttt

SEQ ID NO: 125 - DNA, 17 bp, Pongo pygmaeus
aggaaaggctgagtttt

SEQ ID NO: 126 - DNA, 17 bp, homo sapiens
aagaaaggccgaatttt

SEQ ID NO: 127 - DNA, 14 bp, mus musculus
aaaaggactatttt

SEQ ID NO: 128 - DNA, 15 bp, macaca mulatta
aaagtggcaagtttc

SEQ ID NO: 129 - DNA, 15 bp, homo sapiens
cttccgtgggtttgt

SEQ ID NO: 130 - DNA, 16 bp, Pan troglodytes
cttacgtagggttttt

SEQ ID NO: 131 - DNA, 18 bp, Pongo pygmaeus
agtgttcgttgcgctttt

SEQ ID NO: 132 - DNA, 19 bp, macaca mulatta
gagggtggtttgttgtttt

SEQ ID NO: 133 - DNA, 14 bp, mus musculus
ggggagtttgtttt

SEQ ID NO: 134 - DNA, 15 bp, macaca mulatta
gcgggccgacctttt

SEQ ID NO: 135 - DNA, 15 bp, Pongo pygmaeus
gcgggccgacctttt

SEQ ID NO: 136 - DNA, 15 bp, mus musculus
gcgtgcccacgtttt

SEQ ID NO: 137 - DNA, 15 bp, Pan troglodytes
tgcgatgttactttt

SEQ ID NO: 138 - DNA, 19 bp, Pan troglodytes
tggtgtggtctgttgtttt

SEQ ID NO: 139 - DNA, 21 bp, mus musculus
cgtggtgtgctagttaatttt

SEQ ID NO: 140 - DNA, 21 bp, Pongo pygmaeus
cggggtgttacattgtgtttt

SEQ ID NO: 141 - DNA, 21 bp, Macaca mulatta
tggtcgcgaggcggctctttt

SEQ ID NO: 142 - DNA, 23 bp, Pan troglodytes
cagggaggttatgactaactttt

SEQ ID NO: 143 - DNA, 23 bp, Pongo pygmaeus
cagcgaggtggtgaataactttt

SEQ ID NO: 144 - DNA, 10 bp, mus musculus
cttaactttt

SEQ ID NO: 145 - DNA, 25 bp, Macaca mulatta
aatgatggtgtgatgacaaactttt

SEQ ID NO: 146 - DNA, 19 bp, Macaca mulatta
tggtgaggtctactatttt

SEQ ID NO: 147 - DNA, 21 bp, mus musculus
cgtggtgtgctagttaatttt

SEQ ID NO: 148 - DNA, 20 bp, homo sapiens
cggcgtgattcatacctttt

SEQ ID NO: 149 - DNA, 20 bp, Pan troglodytes
cggcgtgattcacacctttt

SEQ ID NO: 150 - DNA, 21 bp, Pongo pygmaeus
cggggtgttacattgtgtttt

SEQ ID NO: 151 - DNA, 20 bp, Pongo pygmaeus
gaagcgggtgcttacatttt

SEQ ID NO: 152 - DNA, 20 bp, mus musculus
gaagcgggtgcttcactttt

SEQ ID NO: 153 - DNA, 20 bp, Pan troglodytes
gaagcaggtgcttgtatttt

SEQ ID NO: 154 - DNA, 20 bp, macaca mulatta
gaagcaggtgcttctgtctt

(19) United States
(12) Patent Application Publication
(10) Pub. No.: US 2015/0203920 A1
(43) Pub. Date: Jul. 23, 2015

(54) COMPOSITIONS AND METHODS FOR USING TRANSFER RNA FRAGMENTS AS BIOMARKERS FOR CANCER

(71) Applicant: University of Virginia Patent Foundation, Charlottesville, VA (US)

(72) Inventors: Anindya Dutta, Charlottesville, VA (US); Pankaj Kumar, Charlottesville, VA (US)

(73) Assignee: University of Virginia Patent Foundation, Charlottesville, VA (US)

(21) Appl. No.: 14/422,955

(22) PCT Filed: Aug. 20, 2013
(86) PCT No.: PCT/US2013/055776

§ 371 Date: Feb. 20, 2015

Related U.S. Application Data
(60) Provisional application No. 61/691,081, filed on Aug. 20, 2012.

(51) Int. Cl. C12Q 1/68

(52) U.S. Cl. CPC C12Q 1/6886; C12Q 1/6869; C12Q 1/6888; C12Q 2600/178; C12Q 2600/158; C12Q 2600/112

(57) ABSTRACT

Analysis of over 50 short RNA libraries revealed that tRFs are present in all human cell lines and exist in mice, flies, worms, and yeasts. Specific tRNA genes yield tRFs generated by cleavage at sites conserved across different cells within a species, and all three potential tRFs from a given tRNA gene were not always present or equally abundant. tRF-1 and -3 were highly abundant in the cytoplasm, while tRF-5 were mostly in the nucleus. tRF-5 and -3 were found in adult mouse tissues, tRF-1 were relatively rare in adult tissues but in greater amounts in mouse embryos and embryonic stem cells. Several tRF-1 sequences were conserved between mice and humans and expression was tissue-specific. tRFs are shown to be markers for cancer. For example greater amounts of tRF-1 were found in B cell malignancies compared to normal B cell, and tRF-5 and -3 were at higher levels in lung cancer compared to normal lung.

### CROSS REFERENCE TO RELATED APPLICATIONS

This application is entitled to priority pursuant to 35 U.S.C. §119(e) to U.S. provisional patent application No. 61/691,081, filed on Aug. 20, 2012. The entire disclosure of the afore-mentioned patent application is incorporated herein by reference.

### STATEMENT REGARDING FEDERALLY SPONSORED RESEARCH OR DEVELOPMENT

This invention was made with government support under Grant Nos. PO1 CA104106 and R01GM84465 awarded by The National Institutes of Health. The government has certain rights in the invention.

### BACKGROUND

Small RNAs, of which the microRNAs (miRNAs) are the most extensively studied, have emerged as important players in various aspects of biology. The miRNAs constitute a large fraction of small non-coding RNAs, are ˜22-nucleotides (nt) long and are generated endogenously to regulate gene expression at the post-transcriptional level (Bartel 2004; Yekta et al. 2004; Lee and Dutta 2009). A miRNA is usually transcribed as a primary miRNA transcript (pri-miRNA) by RNA Polymerase II (Lee et al. 2004a). The pri-miRNA forms a hairpin-structure that is cleaved by the RNase III enzyme Drosha, with its co-factor DGCR8, to form a hairpin shaped precursor miRNA (pre-miRNA) (Han et al. 2004; Han et al. 2009). The pre-miRNA is exported to the cytoplasm by Exportin-5 (Yi et al. 2003; Lund et al. 2004) where it is cleaved in the loop region by another RNase III, Dicer, to generate a ˜22 nt miRNA:miRNA* duplex (Khvorova et al. 2003). The miRNA:miRNA* then associates with an Argonaute (AGO) protein such that the miRNA strand is stably incorporated, while the miRNA* strand dissociates and is degraded (Khvorova et al., 2003). The miRNAs loaded on the AGO protein are known to show A/U bias at their 5′ terminal base (Mi et al., 2008; Czech and Hannon, 2011).

The other small RNAs, which are widely used in experimental knock down of gene expression, are small interfering RNA (siRNA) that are also generated by sequential processing of double-stranded RNA by Dicer (Babiarz et al., 2008; Eamens et al. 2008). siRNA are generated endogenously in Drosophila (Czech et al., 2008; Okamura et al. 2008), S. pombe (Buhler et al., 2008) and mouse (Babiarz et al., 2008). Drosophila encodes two Dicers, of which Dicer-1 is involved in miRNA biogenesis and Dicer-2 is involved in siRNA biogenesis (Lee et al., 2004b). In addition to Dicer-2, a double stranded RNA binding protein R2D2 and Ago2 are also involved in the biogenesis of siRNA in Drosophila. R2D2 binds Dicer-2 and is required for loading siRNA onto Ago-2 (Czech et al., 2008). However, in humans endogenous siRNA has not been reported.

New species of short RNAs continue to be discovered, e.g. PIWI interacting RNA (piRNA) (Brennecke et al., 2007; Lin, 2007) or repeat associated RNA (rasiRNA) (Aravin et al., 2001; Aravin et al., 2003). The piRNAs are 24-29 base long, germ cell-specific endogenous small RNA (Aravin et al., 2003). Most of these RNA are transcribed from repetitive regions of the genome. The piRNA, which are associated with the Ago homolog PIWI, also show strong preference for “U” at the 5′ end (Kim et al., 2009; Nagao et al., 2010).

Since the discovery of the first small RNA, lin-4, in C. elegans (Lee et al. 1993; Wightman et al. 1993) the number of small RNA has increased substantially in each and every organism (Kozomara and Griffiths-Jones 2011). Considering the importance of small RNA in gene regulation, a number of recent studies were devoted to finding novel non-coding RNA in various species. Technical advancements in sequencing technology have accelerated the discovery of novel small non-coding RNAs.

Recently it was reported that another class of small non-coding RNA that were mapped on tRNA genes, were <˜30 bases long and not generated by cleavage in the anti-codon loop (Lee et al. 2009). The tRFs (transfer RNA related fragments) include three groups or families, those originating from the 5′ and 3′ ends of mature tRNA were called tRF-5 and tRF-3, respectively, whereas those generating from the 3′ trailer regions of precursor tRNAs were called tRF-1 (Lee et al. 2009). tRF-1001, which corresponds to the 3′ trailer sequence of tRNASerTGA, was found to be essential for normal cell proliferation and for passage through the G₂ phase of the cell cycle (Lee et al. 2009). Cole et al. subsequently identified tRNA fragments obtained from the 5′ end of mature tRNA (5-series) from deep sequencing data of small RNAs isolated from HeLa cells (Cole et al. 2009). They further show that tRNA fragments arising from the tRNAGln are generated by Dicer (Cole et al. 2009). Haussecker et al. reported “Type I” (corresponding to tRF-3) and “Type II” (corresponding to tRF-1) tRFs in HEK293 human cell lines (Haussecker et al. 2010). They too report that the generation of tRF-3 is dependent on Dicer (Haussecker et al. 2010). In addition, they report the association of tRFs with Ago 3-4 with experimental re-direction to Ago-2 (Haussecker et al. 2010). Couvillion et al. report tRF-3 of 18-22 nucleotides in Tetrahymena interacting with the Twi12 protein, a Piwi family protein (Couvillion et al. 2010). TRFs interacting with Twi12 protein show “U” bias at their 5′ end (Couvillion et al. 2010). The parallel between the above-described reports on tRFs has been highlighted in recent review on tRFs (Pederson 2010). Because the existence of tRFs and the various tRF families (i.e., tRF-1, tRF-3, and tRF-5) is a recent discovery, much remains to be learned of the function and role of these fragments.

There is a long felt need in the art for compositions and methods useful for studying tRFs and for capitalizing on their function and expression to identify and distinguish normal and aberrant cell processes and to identify and diagnose diseases, disorders, and conditions associated with their expression or change in expression.

### SUMMARY OF THE INVENTION

The present invention provides compositions and methods and biomarkers useful screening for cancers and for evaluation of how well a cancer responds to therapy or for detection of recurrence of cancers. These biomarkers are tRF fragments. In one aspect, a single tRF fragment is useful. In another aspect, a family of tRF fragments is useful. In one embodiment, the present invention encompasses the use of the tRF-5, tRF-3, and tRF-1 groups, alone or in combination, as biomarkers for identifying, diagnosing, monitoring the treatment of, developing treatment strategies, and monitoring the progression of cancer. The invention further encompasses the use of specific tRNA fragments within each group.

Enormous amounts of high-throughput sequence of small RNA libraries from various species have now been reported in various publicly available databases. Disclosed herein is a systematic global analysis of tRFs in the publicly available data to answer the following questions: (1) Are the tRFs limited to only a few cell lines or are they ubiquitous? (2) Are there any other species of tRFs besides tRF-5, 3, and -1? (3) Are they present in other species? (4) How do we differentiate the tRFs from random degradation products of tRNA? (5) Are the tRFs (originating from a particular tRNA) identical across different cell lines? (6) Does the canonical miRNA or siRNA processing machinery have any role in tRF generation? (7) Do the tRFs show differential expression in any disease? These embodiments are addressed in the examples.

The present application discloses that, inter alia, tRF-1, tRF-3, and tRF-5 tRNA fragments are differentially expressed from one another and are found at different levels/amounts in normal cells versus their counterpart malignant/cancer cells. Additionally, depending on the type of cancer, the amount of each varies. Sequences for specific members of the tRF-1, tRF-3, and tRF-5 families used herein, 154 in all, are provided in Table 1 and Supplementary Tables 1, 2, and 3. The tables further provide names for the specific tRNAs. Other useful tRFs are known in the art, for example in Lee et al., 2009, Genes and Development.

The present invention provides for the use of one or more markers for detecting tRFs of the invention, measuring the tRFs to determine the amount of tRFs as a group or individually, and diagnosing cancer cells and cancer based on the amount and type of tRFs measured. In one aspect, one or more tRF markers of the invention can be used alone or in combination. In another aspect, at least two markers (i.e., fragments) of the invention are used. In another aspect, at least 3 markers are used. The present application discloses multiple nucleic acids and sequences and their use, including useful homologs and fragments thereof, for practicing the methods of the invention. In one aspect, one or more fragments of a tRF family are detected and measured. In another aspect, one or more fragments of each of two tRF families are detected and measured. In yet another aspect, one or more fragments of each of three tRF families are detected and measured.

tRF family or group means transfer fragments from a group such as tRF-1, tRF-3, or tRF-5. That is, a family or group comprises multiple fragments (see the definitions of tRF-1, -3, and -5 herein). For example, the tRF-1 family would include multiple individual identified tRF-1 fragments, including those described herein, such as those having SEQ ID NOs:68-99.

In one aspect, the tRFs used are selected from the group consisting of tRF-5, tRF-3, and tRF-1. In one aspect, some useful sequences include SEQ ID NOs:1-99. Other useful sequences included SEQ ID NOs:100-154. Some useful tRF-5s of the invention include, but are not limited to, those tRF-5s having SEQ ID NOs:1-34. Some useful tRF-3s of the invention include, but are not limited to, those tRF-3s having SEQ ID NOs:35-67. Some useful tRF-1s of the invention include, but are not limited to, those tRF-1s having SEQ ID NOs:68-99, 100, 109, 114, 119, 126, 129, and 148. The present invention encompasses the use of different markers and/or different combinations of the markers for identifying and diagnosing different cancers. It will be appreciated that in some cases one tRF fragment is measured. In another case, multiple tRF fragments are measured, for example, to determine the amount of a type of tRF fragment (1 or 3 or 5) present in a cell or tissue.

In one embodiment, the cancer being identified, diagnosed, detected or treated is selected from the group consisting of carcinoma, sarcoma, uterine cancer, ovarian cancer, B cell malignancies, lung cancer, adenocarcinoma, adenocarcinoma of the lung, non-small cell lung cancer, squamous carcinoma, squamous carcinoma of the lung, malignant mixed mullerian tumor, leukemia, lymphoma, osteosarcoma, endometrioid carcinoma, melanoma, breast cancer, prostate cancer, cervical cancer, skin cancer, pancreatic cancer, colorectal cancer, head and neck cancer, liver cancer, pancreatic cancer, esophageal cancer, stomach cancer, endometrial cancer, adrenal cancer, salivary gland cancer, bone cancer, brain cancer, cerebellar cancer, colon cancer, rectal cancer, oronasopharyngeal cancer, bladder cancer, basal cell carcinoma, hard palate carcinoma, squamous cell carcinoma of the tongue, meningioma, pleomorphic adenoma, astrocytoma, chondrosarcoma, cortical adenoma, hepatocellular carcinoma, pancreatic cancer, squamous cell carcinoma, Wilm's tumor, teratocarcinoma, malignant teratoma, mesothelioma, Kaposi's sarcoma, thyroid cancer, neuroblastoma, retinoblastoma, and renal cancer.

In one embodiment, the compositions and methods of the invention are useful for detecting and diagnosing B cell malignancies and for monitoring the progression of such malignancies and their treatment. The present invention further provides for methods to help determine which treatments to use, depending the type and levels of tRFs detected and measured. In one aspect, B cell malignancies have higher amounts of tRF-1 than their normal counterparts. In one aspect, more than one tRF-1 is detected and measured. In one aspect, the tRF-1 amounts are at least five times higher. In another aspect, they are at least 10 times higher. In yet another aspect, they are at least 50 times higher. In yet another aspect, they are at least about 100 times higher. In one aspect, when more than one tRF-1 is detected and measured, the total amount of each is combined. In one aspect, one or more tRF-1 having SEQ ID NOs:68-99 are detected and measured. In one aspect, the amounts are totaled. In one aspect, each of SEQ ID NOs:68-99 are detected and measured. In another aspect, B cell malignancies have normal (similar) amounts of tRF-5 compared to their normal cell counterparts. In another aspect, B cell malignancies have normal amounts of tRF-3 compared to their normal cell counterparts. In another aspect, B cell malignancies have normal amounts of tRF-3 and tRF-5 compared to their normal cell counterparts, but have higher amounts of tRF-1 compared to their normal cell counterparts. Some useful tRF-5 fragments of the invention include those having SEQ ID NO:s1-34. Some useful tRF-3s fragment of the invention include those having SEQ ID NOs:35-67. Some useful tRF-1s of the invention include those having SEQ ID NOs:68-99.

The present application discloses higher levels of both tRF-5 and tRF-3 in lung cancer relative to the amounts found in normal lung tissue. In one embodiment, the compositions and methods of the invention are useful for detecting and diagnosing lung caner and for monitoring the progression of such malignancies and their treatment. The present invention further provides for methods to help determine which treatments to use. In one aspect, lung cancer cells have higher amounts of tRF-5 than their normal counterpart cells. In one aspect, lung cancer cells have higher amounts of tRF-3 than their normal counterpart cells. In one aspect, lung cancer cells have lower amounts of tRF-1 than their normal counterpart cells. In another aspect, lung cancers have higher levels of tRF-5 compared to their normal cell counterparts and higher levels of tRF-3 compared to their normal cell counterparts. In one embodiment, one or more tRF-5 fragments are detected and measured. In one embodiment, one or more tRF-3 fragments are detected and measured. Some useful tRF-5 fragments of the invention include those having SEQ ID NOs:1-34. In one embodiment, all tRF-5s having SEQ ID NOs:1-34 are detected and measured. In one embodiment, the amount of each is totaled. Some useful tRF-3s fragments of the invention include those having SEQ ID NOs:35-67. In one embodiment, all tRF-3s having SEQ ID NOs: 35-67 are detected and measured. In one embodiment, the amount of each is totaled. Some useful tRF-1 s of the invention include those having SEQ ID NOs:68-99.

In one embodiment, lung cancers have higher amounts of both tRF-5 and tRF-3 than their normal counterpart tissue. In one aspect, tRF-1 is not different in tumors relative to normal lung tissue.

The invention provides for detecting and measuring the amount of the fragments and nucleic acids of the invention in a sample. In one aspect, the amounts are useful in detecting or identifying cancer. The results can be compared to a standard or to a sample known to have a certain amount of the marker. The normal or standard samples used for comparison to a test sample can be from the test subject (normal counterpart tissue or cells, etc.) or from a standard containing a known amount of at least one tRF or at least one family of tRFs.

One method of comparison of tRF amounts comprises comparing the amount of reads for a tRF or for a group of tRFs and then comparing the amount for a cancer relative to a standard or to a normal tissue. This can also be used for comparing different cell types, stage of cell differentiation, and species. In one aspect, a tRF is detected and analyzed at about 10 or more reads per million to about 10,000 reads per million. In one aspect, the reads are at least about 5, or 10, or 20, or 100, or 1,000, 5,000, or 10,000 per million. In one aspect, reads are measured and expressed as reads of tRFs per million reads of short RNAs measured.

In one aspect, the amount of reads is normalized.

As disclosed herein, once detected and measured, the difference in amount of each tRF is used to distinguish cancer cells from normal cells. In one aspect, amounts of one or more tRFs are higher in a cancer cell. In one aspect, the increase is by at least 10%. In one aspect, the increase is about five times higher than in a normal cell. In another aspect, the increase is at least about 10, 20, 50, 100, 200, 1,000, or 5,000 times over the amount in a normal cell. In one aspect, the amount of reads is normalized. In one aspect, when two or more tRF fragments of a tRF family are detected and measured, their amounts are totaled and used as one combined number for that family. For example, when comparing tRF-5 amounts in a cancer relative to tRF-5 amounts in a normal counterpart tissue or standard sample, the reads for all of the individual tRF-5 fragments measured in the cancer sample are totaled and that number is compared to the total number of tRF-5 reads for the normal counterpart. In one aspect, one or more individual tRF fragments can be compared to the same one or more individual tRF fragments when detecting, measuring, and comparing the cancer amount to a normal or standard amount.

It is disclosed herein that different cancers have different expression profiles and total amounts of tRF-5, tRF-3, and tRF-1. The present invention provides compositions and methods useful for detecting cancer cells by detecting, measuring, and comparing tRFs in a tissue or cell suspected of being cancerous to the same tRFs in a normal sample or to a standard. As disclosed herein, in one aspect, one family of tRFs may be present in greater amounts in a cancer relative to normal tissue or cells. In another cancer, the same tRFs may be lower. One of ordinary skill in the art will appreciate that the compositions and methods of the invention are useful for establishing a database of normal amounts of tRFs expressed in a tissue or cell and using those amounts for comparison to the amounts found in a cancer. In one aspect, the total amounts of two groups of tRFs are higher in a cancer. In one aspect, the total amounts of all three groups of tRFs are higher in a cancer. In another aspect, the total amounts of two groups of tRFs are lower in a cancer. One of ordinary skill in the art will appreciate, that as disclosed herein, not all cancers will be the same but that the compositions and methods of the invention can still be useful when at least one individual tRF of a group is different in the cancer relative to the normal counterpart.

In one embodiment, the tRFs can be compared using a heat map. In one aspect, the comparison is made using the z-score of a heat map.

In one embodiment, the sample is selected from the group consisting of tumor biopsy, tissue sample, blood, plasma, peritoneal fluid, follicular fluid, ascites, urine, feces, saliva, mucus, phlegm, sputum, tears, cerebrospinal fluid, effusions, lavage, and Pap smears. In one aspect, the sample is blood. In one aspect, the sample is serum. In one aspect, the sample is plasma.

In one embodiment, diagnosis of cancer made by measuring a tRF or tRF family of the invention is used to aid in establishing a treatment or treatment regimen for a subject with cancer. The present invention provides compositions and methods useful for personalized medicine. In one embodiment, the present invention provides compositions and methods useful for selecting a subject with cancer who will be responsive to treatment with a regulator of tRF-5, -3, or -1, comprising detecting and measuring the amount of tRF-5, -3, or -1 in a sample from the subject, wherein the amount of tRF-5, -3, or -1 in the sample indicates that the subject will be responsive to treatment with a regulator of tRF-5, -3, or -1. A regulator of tRF is an agent useful for regulating the expression or function of a tRF.

The present invention also provides compositions and methods useful for preventing and for treating cancer based on the amounts of tRF-5, -3, or -1 detected and measured.

The invention further provides kits for diagnosing, detecting, imaging, and treating cancers based on the levels of tRF-5, -3, or -1.

In one embodiment, the present invention provides for the use of the nucleic acids and sequences of Table 1 and Supplementary Tables 1-3, as well as useful fragments and homologs thereof.

It is disclosed herein that different cell types have different expression profiles for tRF-1, tRF-3, and tRF-5. Further disclosed herein is that the cell profile can vary based on the differentiation stage of the cell. It is disclosed herein that different tissues have different expression profiles for tRF-1, tRF-3, and tRF-5. In one embodiment, tRFs of the invention are useful for identifying and distinguishing different cell types from one another. In one embodiment, tRFs of the invention are useful for distinguishing different tissues from one another. In one embodiment, the compositions and methods of the invention are useful for distinguishing adult tissue from embryonic tissue. In another aspect, cells or tissues from different species can be distinguished based on their tRF expression profiles.

It is known in the art and further disclosed herein that tRFs can vary in their length. The present invention is not limited by the particular length of a tRF, and can, for example, include the use of, detection of, and measurement of single tRFs having a size ranging, for example, from about 5 nucleotide residues to about 40 nucleotide residues. In one aspect, the length ranges from about 10 nucleotide residues to about 35 nucleotide residues. In another aspect, the length ranges from about 15 nucleotide residues to about 30 nucleotide residues.

The present invention further provides a kit for detecting and measuring tRFs, including tRF-5s, tRFs-3s, and tRF-1s, for use in detecting and diagnosing cancer and for distinguishing cell types, cell differentiation states, and cells of different species, comprising reagents, polynucleotides, an applicator, and an instructional material for the use thereof.

Various aspects and embodiments of the invention are described in further detail below.

### BRIEF DESCRIPTION OF THE DRAWINGS

FIG. 1: Non-random mapping of small RNA (tRFs) on tRNA genes (HEK293 human cell line). (A) tRNA gene co-ordinates were collapsed to 1-73 bases long mature tRNA. The scale 1 to 73 on the x-axis is the 1st to 73rd base of mature tRNA gene. The 5′ and 3′ ends of tRFs mapped on tRNA were recorded. The number of tRF ends that map to a specific base of tRNA locus is shown. The dotted lines predict the three types of tRFs. (B) Frequency of the three types of tRF in different human cell lines. tRF alignments that start with 1st or 2nd base of tRNA were collated as tRF-5 and whose 3′ end mapped to 3′ end of tRNA and have a CCA at their 3′ end were categorized as tRF-3. tRFs whose 5′ end matched with the first or second bases of 3′ trailer sequence of a tRNA were categorized as tRF-1. The number of tRF-5, tRF-3, and tRF-1 mapped in each cell line was normalized with the total number of reads in the analyzed library. Cell lines include—HEK293, HeLa, U20S, 143B, A549, H520, SW480, DLD2, MCF7, and MDA231.

FIG. 2: (A) Length distribution of tRF-5, tRF-3, and tRF-1 in HEK293 human cell line plotted against total number of reads of that tRF. Each species of tRF was grouped into individual bins. The number of tRFs of a specific length observed for each of tRF-5, tRF-3, and tRF-1 is shown here. (B) Length distribution of tRFs that had at least 20 reads per million plotted against number of unique tRFs of a particular length. (C) The different cut sites defined on mature tRNA on the basis of the length of tRF-5 and -3 in human. Three sub-species of tRF-5, corresponding to peaks at: 15 bases (tRF-5a), 22 bases (tRF-5b) and 31 bases (tRF-5c) were observed. The two sub species of tRF-3 were of 18 (tRF-3a) and 22(tRF-3b) bases long. (D) Precise cut sites generate specific tRFs: tRF-5 of GlyGCC, tRF-3 of ValCAC and tRF-1 of LeuTAG tRNA. The tRNAs analyzed are different for each panel since a particular tRNA does not give rise to all the tRF series.

FIG. 3: Non-random mapping of small RNA (tRFs) on tRNA genes of other species. The x-axis corresponds to the tRNA genes as explained in FIG. 1. The number of tRF ends (5′ or 3′) mapped at each base given as reads per million in: (A) mouse embryonic stem cells, (B) mouse cell line NIH3T3, (C) D. melanogaster, (D) C. elegans, (E) S. cerevisiae and (F) S. pombe. (G) Shows the frequency of tRF-5, tRF-3, and tRF-1 in each species. (H) The computational prediction of length distribution of tRF-1 in human, mouse, Drosophila, C. elegans, S. cerevisiae, and S. pombe.

FIG. 4: A given tRNA does not yield tRF-5, 3, and -1 at equal abundance. Number of reads per million of specific tRF-5, tRF-3 and tRF-1 is shown. The tRNA gene were selected on the basis of tRF-1 that had >20 reads per million in HEK293 human cell line library. The duplicate tRNA genes (tRNA codes for same anticodon) are marked with special character “*”, “#”, “$”, “%” and &. In the case of duplicate tRNA genes the tRF-1 abundance is different for individual tRNA genes, but the tRF-5 and tRF-3 abundance is the same in duplicates because of the high sequence conservation of mature tRNAs with the same anticodon.

FIG. 5: (A) A/U bias at the 5′ end of tRF-3. tRF-3 is generated by a cleavage between A/U-A/U bases. An “A” or “U” bias was present at the 5′ terminus (+1) as well as at the immediate upstream base (−1) of the most abundant tRF-3 mapped on an individual tRNA gene family in human HEK293 cell line (Mayr and Bartel 2009), mouse tissue (Chiang et al. 2010), and Drosophila (Ameres et al. 2010). (B) 3′ ends of tRF-5 indicated that “G” or “C” was more abundant compared to “A” or “U” at the 3′ end of tRF-5 in human HEK293 human cell line, mouse tissue sample, and Drosophila. The immediate downstream base was mostly “A” or “U” in human and mouse. Interestingly in the drosophila downstream base to tRF-5 showed strong bias for “G” or “C”. −1 is the 3′ end of the tRF-5 and +1 is the base immediately downstream from the cleavage site that generates the 3′ end.

FIG. 6: Processing of tRFs is independent of Dicer or DGCR8 and tRFs mostly do not associate with Ago1/2 protein. (A) Mutation of Dicer or DGCR8 did not decrease the expression of all three tRFs in mouse embryonic stem cells. (B) In contrast nearly hundred-fold suppression of the sequencing frequency of several microRNAs was observed in Dicer or DGCR8 knock out mouse embryonic stem cell. (C) TRF abundance is either increased or unchanged in Dicer mutant in S. pombe. (D and E) A similar trend of increased abundance of tRFs was also observed in Dicer-1, Dicer-2, and R2D2 mutants of D. melanogaster. (F) The miRNA expression was decreased in Dicer-1 mutant compared to wild type strain and this decrease in miRNA expression was not observed in Dicer-2 mutant. (G) & (I) Less than 2% of the tRFs are associated with Ago-1/2 protein in human (G) and mouse (I). (H) (J) In contrast significant amount of miRNA (80% of mir-21 in human) was associated with Ago 1/2 protein in human HeLa cell (H) and in mouse NIH3T3 cell lines (J) in the same experiment.

FIG. 7: Cytoplasmic vs. Nuclear abundance of tRFs. (A) Human HeLa cell line: tRF-5 is mostly present in nucleus whereas tRF-3 and tRF-1 are mostly enriched in cytoplasm. (B-D) tRF expression in different mouse tissues and embryonic stem cells (ESC).

FIG. 8: tRF-1 are increased in malignant B cells. (A-C) The abundance of tRFs in normal B-cells and related malignant B-cells in different B-cell subsets is shown. (A) naïve B cells, (B) plasma B cell and (C) germinal center B cell. (D-E) The individual tRF-1 (read number >20 per million) are increased in the malignant B-cells. (D) Germinal center B cells and malignant counterpart. (E) Plasma B cell and malignant counterpart.

FIG. 9: Expression patterns of tRF-1 in human cell lines and tissues. Each row represents the relative expression levels of a single tRF-1 and each column shows the expression levels of different tRF-1 for an individual sample. OS=Osteosarcoma, FB=Fibroblast and PBMC=peripheral blood mononuclear cell.

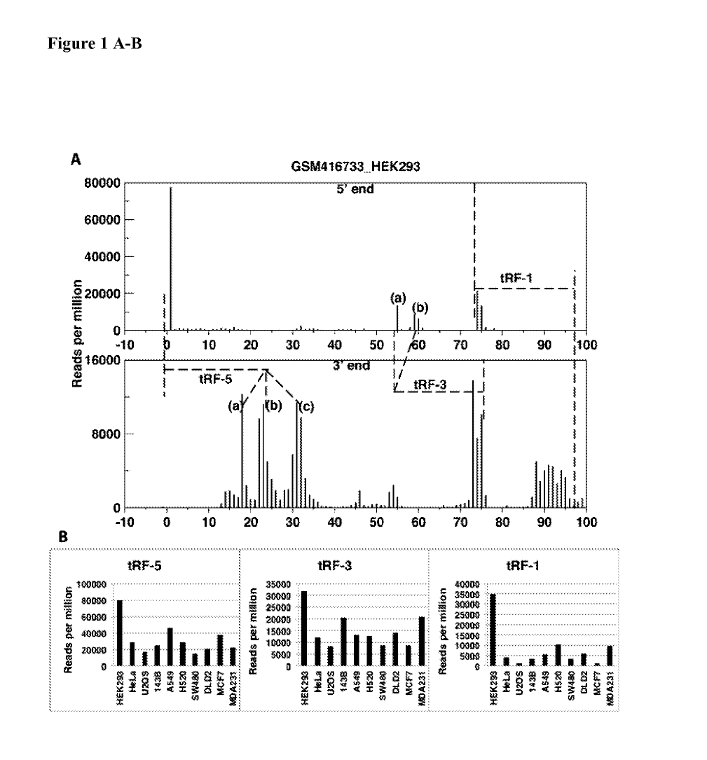

Supplementary FIG. 1: Non-random mapping of small RNA (tRFs) on tRNA genes in various human cell lines. The axes and other details are same as given in FIG. 1 legend.

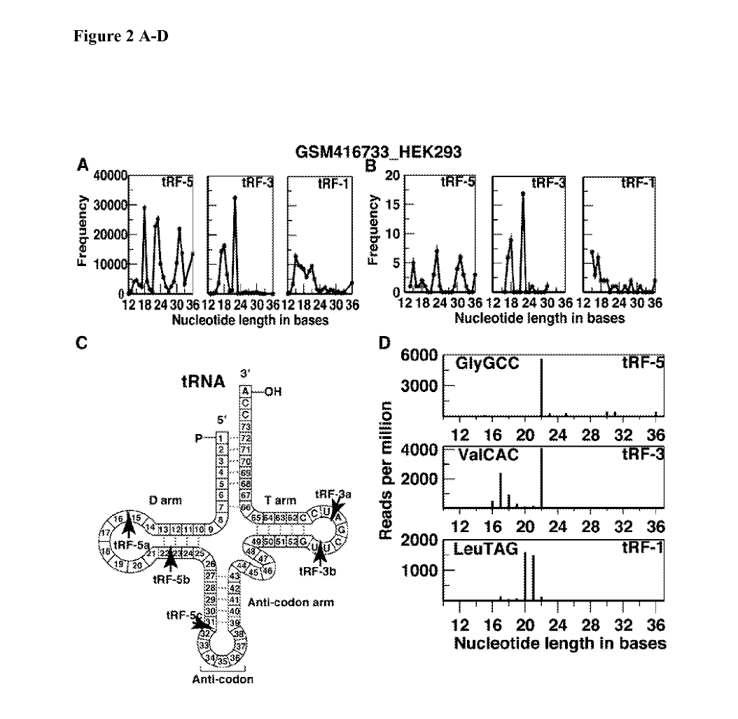

Supplementary FIG. 2: Length distribution of tRF-5, 3, and -1 in various human cell lines. The axes and other details are same as given in FIG. 2 legend.

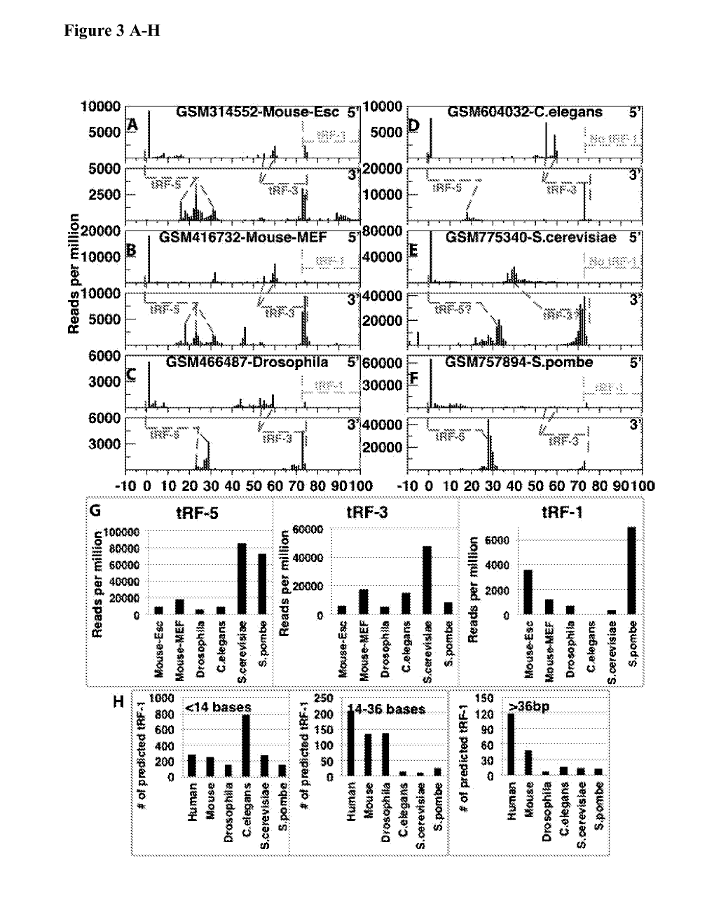

Supplementary FIG. 3: Precise cut sites generate specific tRFs: tRF-5 of GlyGCC, tRF-3 of ValCAC and tRF-1 of LeuTAG tRNA was extracted and the length distribution of tRF-5, 3, and -1 is shown for HeLa, 143B, SW480 and MCF7 human cell lines.

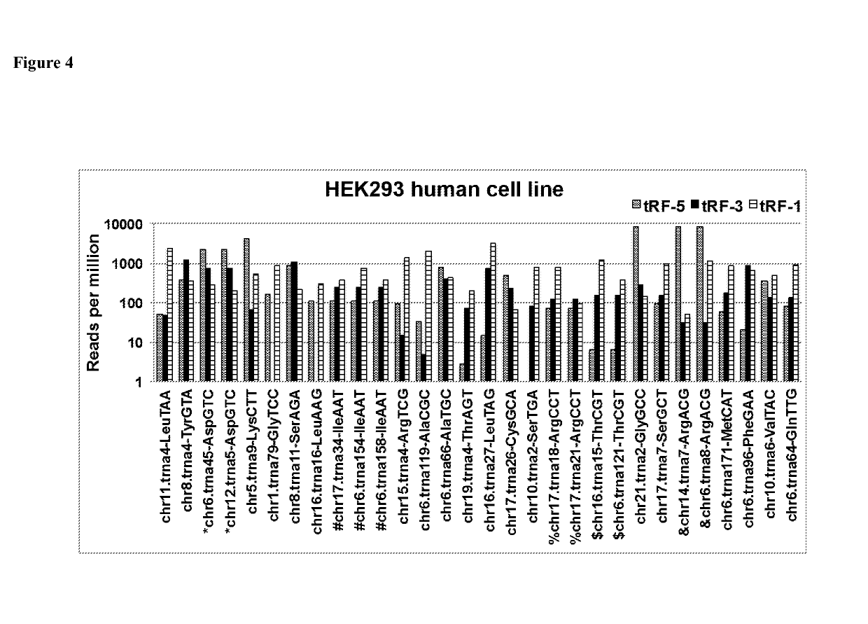

Supplementary FIG. 4: tRF-5 and tRF-3 are equally abundant in normal and cancer B cells.

Example 2, FIG. 1: tRF-5 and -3 are increased and tRF-1 decreased in several human lung carcinomas compared to normal adjoining lung. The abundance of tRFs in normal lung tissue and carcinoma (expressed as reads of tRFs/million reads of short RNAs) is shown. A subset of tRF-5 and -3 are 10-20 fold higher in several tumors compared to normal. Ad=Adenocarcinoma; Sq=Squamous Cell carcinoma.

tRF-1 is not increased in lung cancers—Data Source: National Center for Biotechnology Information/National Library of Medicine/National Institutes of Health website of the U.S. government-acc=GSE33858 (Unpublished).

Example 2, FIG. 2: tRF-5 abundance is increased in human lung carcinomas compared to normal adjoining lung. The abundance of tRF-5s in normal lung tissue and carcinoma (expressed as number of reads of tRF-5s/million reads of short RNAs) is shown. All tRF-5s are considered together. Box and whiskers plot shows the median and interquartile range for the data. Asterisks indicate outliers. The difference in expression levels is statistically significant (p-value 0.0019) which was calculated by paired t-test. Data Source: National Center for Biotechnology Information/National Library of Medicine/National Institutes of Health website of the U.S. government-acc=GSE33858 (Unpublished).

Example 2, FIG. 3: tRF-3 abundance is increased in human lung carcinomas compared to normal adjoining lung. The abundance of tRF-3s in normal lung tissue and carcinoma (expressed as number of reads of tRF-3s/million reads of short RNAs) is shown. All tRF-3s are considered together. Box and whiskers plot shows the median and interquartile range for the data. Asterisks indicate outliers. The difference in expression levels is statistically significant (p-value 0.0134) which was calculated by paired t-test. Data Source: National Center for Biotechnology Information/National Library of Medicine/National Institutes of Health website of the U.S. government-acc=GSE33858 (Unpublished).

Example 2, FIG. 4: tRF-1 abundance is decreased in human lung carcinomas compared to normal adjoining lung. The abundance of tRF-1 s in normal lung tissue and carcinoma (expressed as number of reads of tRF-1s/million reads of short RNAs) is shown. All tRF-1 s are considered together. Box and whiskers plot shows the median and interquartile range for the data. Asterisks indicate outliers. The difference in expression levels is statistically significant (p-value 0.0211) which was calculated by paired t-test. Data Source: National Center for Biotechnology Information/National Library of Medicine/National Institutes of Health website of the U.S. government-acc=GSE33858 (Unpublished).

Table 1 and Supplementary Tables 1-3 summarize the 154 sequences provided herein and also provide the SEQ ID NOs: for each of the sequences.

### DETAILED DESCRIPTION

### Abbreviations and Acronyms

AGO—Argonaute protein
FB—fibroblast
miRNA—microRNA
NCBI—National Center for Biotechnology Information
nt—nucleotide
Os—osteosarcoma
PBMC—peripheral blood mononuclear cell
piRNA—PIWI interacting RNA
pri-miRNA—primary miRNA transcript
rasiRNA—repeat associated RNA
RNA pol III—RNA polymerase III
RPM—reads per million
siRNA—small interfering RNA
tRF—tRNA related fragment
tRF-1—transfer RNA related fragments generating from the 3′ trailer regions of precursor tRNAs
tRF-3—transfer RNA related fragments originating from the 3′ ends of mature tRNA
tRF-5—transfer RNA related fragments originating from the 5′ ends of mature tRNA

### DEFINITIONS

In describing and claiming the invention, the following terminology will be used in accordance with the definitions set forth below. Unless defined otherwise, all technical and scientific terms used herein have the commonly understood meaning by one of ordinary skill in the art to which the invention pertains. Although any methods and materials similar or equivalent to those described herein may be useful in the practice or testing of the present invention, preferred methods and materials are described below. Specific terminology of particular importance to the description of the present invention is defined below.

The articles “a” and “an” are used herein to refer to one or to more than one (i.e., to at least one) of the grammatical object of the article. By way of example, “an element” means one element or more than one element.

The term “about” as used herein, means approximately, in the region of, roughly, or around. When the term “about” is used in conjunction with a numerical range, it modifies that range by extending the boundaries above and below the numerical values set forth. For example, in one aspect, the term “about” is used herein to modify a numerical value above and below the stated value by a variance of 20%.

The terms “abundance” and “amount” are used interchangeably herein.

As used herein, the term “adjacent” is used to refer to nucleotide sequences which are directly attached to one another, having no intervening nucleotides. By way of example, the pentanucleotide 5′-AAAAA-3′ is adjacent to the trinucleotide 5′-TTT-3′ when the two are connected thus: 5′-AAAAATTT-3′ or 5′-TTTAAAAA-3′, but not when the two are connected thus: 5′-AAAAACTTT-3′.

A disease, disorder, or condition is “alleviated” if the severity of a symptom of the disease or disorder, the frequency with which such a symptom is experienced by a patient, or both, are reduced.

The term “alterations in peptide structure” as used herein refers to changes including, but not limited to, changes in sequence, and post-translational modification.

As used herein, “amino acids” are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

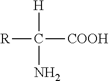

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

The terms “amount” and “abundance” are used interchangeably herein.

“Amplification” refers to any means by which a polynucleotide sequence is copied and thus expanded into a larger number of polynucleotide molecules, e.g., by reverse transcription, polymerase chain reaction, and ligase chain reaction.

As used herein, an “analog” of a chemical compound is a compound that, by way of example, resembles another in structure but is not necessarily an isomer (e.g., 5-fluorouracil is an analog of thymine).

The term “analyte”, as used herein, refers to any material or chemical substance subjected to analysis. In one aspect, the material is a peptide or mixture of peptides. In another aspect, the term refers to a mixture of biomolecules, including, but not limited to, lipids, carbohydrates, and nucleic acids such as DNA and RNA.

The term “anchor”, as used herein, means to purify DNA or cDNA from a particular part of the genome so that the subsequent steps (in this case, ultrahigh throughput paired-end-sequencing) can be restricted to that particular part of the genome. This allows more samples to be covered than if the whole genome was processed. The present applications discloses a novel method of anchoring that can be used for other applications as well, not just identifying structural variations in the genome.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)2, as well as single chain antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

A first nucleic acid region and a second nucleic acid region are “arranged in an antiparallel fashion” if, when the first region is fixed in space and extends in a direction from its 5′-end to its 3′-end, at least a portion of the second region lies parallel to the first strand and extends in the same direction from its 3′-end to its 5′-end.

As used herein, the term “antisense oligonucleotide” means a nucleic acid polymer, at least a portion of which is complementary to a nucleic acid which is present in a normal cell or in an affected cell. The antisense oligonucleotides of the invention include, but are not limited to, phosphorothioate oligonucleotides and other modifications of oligonucleotides. Methods for synthesizing oligonucleotides, phosphorothioate oligonucleotides, and otherwise modified oligonucleotides are well known in the art (U.S. Pat. No. 5,034,506; Nielsen et al., 1991, Science 254: 1497).

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

An “aptamer” is a compound that is selected in vitro to bind preferentially to another compound (for example, the identified proteins herein). Often, aptamers are nucleic acids or peptides because random sequences can be readily generated from nucleotides or amino acids (both naturally occurring or synthetically made) in large numbers but of course they need not be limited to these.

The term “basic” or “positively charged” amino acid as used herein, refers to amino acids in which the R groups have a net positive charge at pH 7.0, and include, but are not limited to, the standard amino acids lysine, arginine, and histidine.

The term “biocompatible”, as used herein, refers to a material that does not elicit a substantial detrimental response in the host.

As used herein, the term “biologically active fragments” or “bioactive fragment” of the polypeptides encompasses natural or synthetic portions of the full length protein that are capable of specific binding to their natural ligand or of performing the function of the protein.

The term “biomolecule”, as used herein, refers broadly to, inter alia, a molecule produced or used by a living organism, or which is a substituent of a living organism. Biomolecules can be natural or synthetic. Biomolecules, include for example, but are not limited to, lipids, carbohydrates, proteins, peptides, and nucleic acids such as DNA and RNA.

The term “cancer”, as used herein, is defined as proliferation of cells whose unique trait—loss of normal controls—results in unregulated growth, lack of differentiation, local tissue invasion, and metastasis. Examples include but are not limited to, melanoma, breast cancer, prostate cancer, ovarian cancer, uterine cancer, cervical cancer, skin cancer, pancreatic cancer, colorectal cancer, renal cancer and lung cancer.

The terms “cell,” “cell line,” and “cell culture” as used herein may be used interchangeably. All of these terms also include their progeny, which are any and all subsequent generations. It is understood that all progeny may not be identical due to deliberate or inadvertent mutations.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. As used herein, the terms “complementary” or “complementarity” are used in reference to polynucleotides (i.e., a sequence of nucleotides) related by the base pairing rules. For example, for the sequence “A G T,” is complementary to the sequence “T C A.” Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “compound,” as used herein, refers to a protein, polypeptide, an isolated nucleic acid, or other agent used in the method of the invention.

As used herein, the term “conservative amino acid substitution” is defined herein as an amino acid exchange within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln;

III. Polar, positively charged residues:
His, Arg, Lys;

IV. Large, aliphatic, nonpolar residues:
Met Leu, Ile, Val, Cys

V. Large, aromatic residues:
Phe, Tyr, Trp

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed. An “otherwise identical sample” means that, for example, when a cancer sample has been obtained, that a control sample would be from adjacent non-cancerous tissue or similar tissue or sample from a subject who does not have cancer.

A “test” cell, tissue, sample, or subject is one being examined or treated.

A “pathoindicative” cell, tissue, or sample is one which, when present, is an indication that the animal in which the cell, tissue, or sample is located (or from which the tissue was obtained) is afflicted with a disease or disorder. By way of example, the presence of one or more breast cells in a lung tissue of an animal is an indication that the animal is afflicted with metastatic breast cancer.

A tissue “normally comprises” a cell if one or more of the cell are present in the tissue in an animal not afflicted with a disease or disorder.

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence polarization or altered light scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

An “enhancer” is a DNA regulatory element that can increase the efficiency of transcription, regardless of the distance or orientation of the enhancer relative to the start site of transcription.

As used herein, an “essentially pure” preparation of a particular protein or peptide is a preparation wherein at least about 95%, and preferably at least about 99%, by weight, of the protein or peptide in the preparation is the particular protein or peptide.

As used in the specification and the appended claims, the terms “for example,” “for instance,” “such as,” “including” and the like are meant to introduce examples that further clarify more general subject matter. Unless otherwise specified, these examples are provided only as an aid for understanding the invention, and are not meant to be limiting in any fashion.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, a “functional” biological molecule is a biological molecule in a form in which it exhibits a property or activity by which it is characterized. A functional enzyme, for example, is one which exhibits the characteristic catalytic activity by which the enzyme is characterized.

A “genomic DNA” of a human patient is a DNA strand which has a nucleotide sequence homologous with a gene of the patient. By way of example, both a fragment of a chromosome and a cDNA derived by reverse transcription of a human mRNA are genomic DNAs.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity” when comparing sequences.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein “injecting or applying” includes administration of a compound of the invention by any number of routes and means including, but not limited to, topical, oral, buccal, intravenous, intramuscular, intra arterial, intramedullary, intrathecal, intraventricular, transdermal, subcutaneous, intraperitoneal, intranasal, enteral, topical, sublingual, vaginal, ophthalmic, pulmonary, or rectal means.

As used herein, an “instructional material” includes a publication, a recording, a diagram, or any other medium of expression which can be used to communicate the usefulness of the compositions and methods of the invention in the kit for identifying and monitoring structural variations in a chromosome. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically binds to a target compound. A ligand (e.g., an antibody) “specifically binds to” or “is specifically immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow and Lane, 1988, Antibodies, A Laboratory Manual, Cold Spring Harbor Publications, New York, for a description of immunoassay formats and conditions that can be used to determine specific immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions.

The term “mass tag”, as used herein, means a chemical modification of a molecule, or more typically two such modifications of molecules such as peptides, that can be distinguished from another modification based on molecular mass, despite chemical identity.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “method of identifying peptides in a sample”, as used herein, refers to identifying small and large peptides, including proteins.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample.

A first nucleic acid region and a second nucleic acid region are “arranged in a parallel fashion” if, when the first region is fixed in space and extends in a direction from its 5′-end to its 3′-end, at least a portion of the second region lies parallel to the first strand and extends in the same direction from its 5′-end to its 3′-end.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

As used herein, a “peptide” encompasses a sequence of 2 or more amino acid residues wherein the amino acids are naturally occurring or synthetic (non naturally occurring) amino acids covalently linked by peptide bonds. No limitation is placed on the number of amino acid residues which can comprise a protein's or peptide's sequence. As used herein, the terms “peptide,” polypeptide,” and “protein” are used interchangeably. Peptide mimetics include peptides having one or more of the following modifications:

1. peptides wherein one or more of the peptidyl C(O)NR linkages (bonds) have been replaced by a non peptidyl linkage such as a CH2 carbamate linkage

(CH2OC(O)NR), a phosphonate linkage, a CH2 sulfonamide (CH 2 S(O)2NR) linkage, a urea (NHC(O)NH) linkage, a CH2 secondary amine linkage, or with an alkylated peptidyl linkage (C(O)NR) wherein R is C1 C4 alkyl;

2. peptides wherein the N terminus is derivatized to a NRR1 group, to a NRC(O)R group, to a NRC(O)OR group, to a NRS(O)2R group, to a NHC(O)NHR group where R and R1 are hydrogen or C1 C4 alkyl with the proviso that R and R1 are not both hydrogen;

3. peptides wherein the C terminus is derivatized to C(O)R2 where R 2 is selected from the group consisting of C1 C4 alkoxy, and NR3R4 where R3 and R4 are independently selected from the group consisting of hydrogen and C1 C4 alkyl.

Synthetic or non naturally occurring amino acids refer to amino acids which do not naturally occur in vivo but which, nevertheless, can be incorporated into the peptide structures described herein. The resulting “synthetic peptide” contains amino acids other than the 20 naturally occurring, genetically encoded amino acids at one, two, or more positions of the peptides. For instance, naphthylalanine can be substituted for tryptophan to facilitate synthesis. Other synthetic amino acids that can be substituted into peptides include L hydroxypropyl, L 3,4 dihydroxyphenylalanyl, alpha amino acids such as L alpha hydroxylysyl and D alpha methylalanyl, L alpha. methylalanyl, beta. amino acids, and isoquinolyl. D amino acids and non naturally occurring synthetic amino acids can also be incorporated into the peptides. Other derivatives include replacement of the naturally occurring side chains of the 20 genetically encoded amino acids (or any L or D amino acid) with other side chains.

The term “peptide mass labeling”, as used herein, means the strategy of labeling peptides with two mass tag reagents that are chemically identical but differ by a distinguishing mass.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

A “polylinker” is a nucleic acid sequence that comprises a series of three or more different restriction endonuclease recognitions sequences closely spaced to one another (i.e. less than 10 nucleotides between each site).

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Plurality” means at least two.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

“Recombinant polynucleotide” refers to a polynucleotide having sequences that are not naturally joined together. An amplified or assembled recombinant polynucleotide may be included in a suitable vector, and the vector can be used to transform a suitable host cell. A recombinant polynucleotide may serve a non-coding function (e.g., promoter, origin of replication, ribosome-binding site, etc.) as well.

A “recombinant polypeptide” is one which is produced upon expression of a recombinant polynucleotide.

A “sample,” as used herein, refers preferably to a biological sample from a subject, including, but not limited to, normal tissue samples, diseased tissue samples, biopsies, blood, saliva, feces, cerebrospinal fluid, semen, tears, and urine. A sample can also be any other source of material obtained from a subject which contains cells, tissues, or fluid of interest. A sample can also be obtained from cell or tissue culture. One of ordinary skill in the art will recognize that such a sample may comprise a complex mixture of peptides.

As used herein, the term “secondary antibody” refers to an antibody that binds to the constant region of another antibody (the primary antibody).

As used herein, the term “solid support” relates to a solvent insoluble substrate that is capable of forming linkages (preferably covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

By the term “specifically binds,” as used herein, is meant an antibody or compound which recognizes and binds a molecule of interest (e.g., an antibody directed against a polypeptide of the invention), but does not substantially recognize or bind other molecules in a sample.

The term “standard,” as used herein, refers to something used for comparison. For example, a standard can be a known standard agent or compound which is administered or added to a control sample and used for comparing results when measuring said compound in a test sample. Standard can also refer to an “internal standard,” such as an agent or compound which is added at known amounts to a sample and is useful in determining such things as purification or recovery rates when a sample is processed or subjected to purification or extraction procedures before a marker of interest is measured. Standard can also refer to a standard sample which is used for comparison to a test sample.

By “structural variation in a chromosome” is meant a change such as an insertion, deletion, translocation, and copy number changes relative to what is considered normal DNA.

A “subject” of analysis, diagnosis, or treatment is an animal. Such animals include mammals, including humans. Non-human animals include, for example, pets and livestock, such as ovine, bovine, equine, porcine, canine, feline and murine mammals, as well as reptiles, birds and fish. The term “pets” refers to dogs, cats, marmosets, hamster, etc. Lower organisms are also included, for example, yeast.

As used herein, a “substantially homologous amino acid sequences” includes those amino acid sequences which have at least about 95% homology, preferably at least about 96% homology, more preferably at least about 97% homology, even more preferably at least about 98% homology, and most preferably at least about 99% or more homology to an amino acid sequence of a reference antibody chain Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the present invention.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. Preferably, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; preferably in 7% (SDS), 0.5 M NaPO₄, 1 mM EDTA at 50° C., with washing in 1×SSC, 0.1% SDS at 50° C.; preferably 7% SDS, 0.5 M NaPO₄, 1 mM EDTA at 50° C. with washing in 0.5×SSC, 0.1% SDS at 50° C.; and more preferably in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984 Nucl. Acids Res. 12:387), and the BLASTN or FASTA programs (Altschul et al., 1990 Proc. Natl. Acad. Sci. USA. 1990 87:14:5509-13; Altschul et al., J. Mol. Biol. 1990 215:3:403-10; Altschul et al., 1997 Nucleic Acids Res. 25:3389-3402). The default settings provided with these programs are suitable for determining substantial similarity of nucleic acid sequences for purposes of the present invention.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the term “transgene” means an exogenous nucleic acid sequence comprising a nucleic acid which encodes a promoter/regulatory sequence operably linked to nucleic acid which encodes an amino acid sequence, which exogenous nucleic acid is encoded by a transgenic mammal

As used herein, the term “transgenic mammal” means a mammal, the germ cells of which comprise an exogenous nucleic acid.

As used herein, a “transgenic cell” is any cell that comprises a nucleic acid sequence that has been introduced into the cell in a manner that allows expression of a gene encoded by the introduced nucleic acid sequence.

The term to “treat,” as used herein, means reducing the frequency with which symptoms are experienced by a patient or subject or administering an agent or compound to reduce the frequency with which symptoms are experienced.

A “prophylactic” treatment is a treatment administered to a subject who does not exhibit signs of a disease or exhibits only early signs of the disease for the purpose of decreasing the risk of developing pathology associated with the disease.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

Methods useful for carrying out the present invention are described herein or are known in the art.

### EMBODIMENTS

The present invention provides compositions and methods to diagnosis cancer based on the unexpected result that various tRFs are differentially expressed in cancers,

Cancer Diagnosis

Detection and diagnosis of cancers based on the level or expression of one or more of tRF-5, -3, and -1 can be performed by obtaining samples from a subject and determining whether the sample is positive, negative, or has lower levels for tRF-5, -3, and -1 and compositions and methods are also provided for in vivo imaging of tRF-5, -3, and -1 varied cells.

In one embodiment, tumors expressing tRF-5, -3, and -1 can be directly targeted for diagnosis. This can be done for example using antibodies or fragments thereof that are directed against and which have been conjugated to an imaging agent useful for in vivo imaging.

In one embodiment, tissue samples and other samples obtained from a subject can be used to detect one or more tRFs. Tissue samples can include tumor biopsies and other tissues where secretions, excretions, or debris from cancer cells, including surface proteins or membranes shed from dead cancer cells. The samples other than tumor biopsies include, but are not limited to, tissue samples, blood, plasma, peritoneal fluids, ascites, follicular fluid, urine, feces, saliva, mucus, phlegm, sputum, tears, cerebrospinal fluid, effusions such as lung effusions, lavage, and Pap smears.

In one embodiment, the cancer is selected from the group consisting of lung cancer, MMMT, bladder cancer, ovarian cancer, uterine cancer, endometrial cancer, breast cancer, head and neck cancer, liver cancer, pancreatic cancer, esophageal cancer, stomach cancer, cervical cancer, prostate cancer, adrenal cancer, lymphoma, leukemia, salivary gland cancer, bone cancer, brain cancer, cerebellar cancer, colon cancer, rectal cancer, colorectal cancer, oronasopharyngeal cancer, NPC, kidney cancer, skin cancer, melanoma, basal cell carcinoma, hard palate carcinoma, squamous cell carcinoma of the tongue, meningioma, pleomorphic adenoma, astrocytoma, chondrosarcoma, cortical adenoma, hepatocellular carcinoma, pancreatic cancer, squamous cell carcinoma, and adenocarcinoma.

In one aspect, the cancer is a metastatic cancer.

The invention is also useful for comparing the levels of a tRF of the invention being imaged to help determine whether a cancer is benign or malignant, based on the level of imaging agent detected (a measure of the amount of the expression, amount or identity).

The invention is also useful for determining the stage of carcinogenesis of a cancer and monitoring its progression from early to late stage cancer. This method is useful for determining the type and amount of therapy to use.

A cancer may belong to any of a group of cancers which have been described. Examples of such groups include, but are not limited to, leukemias, lymphomas, meningiomas, mixed tumors of salivary glands, adenomas, carcinomas, adenocarcinomas, sarcomas, dysgerminomas, retinoblastomas, Wilms' tumors, neuroblastomas, melanomas, and mesotheliomas.

Pharmaceutical Compositions and Administration

The present invention is also directed to pharmaceutical compositions comprising the compounds of the present invention. More particularly, such compounds can be formulated as pharmaceutical compositions using standard pharmaceutically acceptable carriers, fillers, solublizing agents and stabilizers known to those skilled in the art.

The invention is also directed to methods of administering the compounds of the invention to a subject. In one embodiment, the invention provides a method of treating a subject by administering compounds identified using the methods of the invention description. Pharmaceutical compositions comprising the present compounds are administered to a subject in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal means.

In accordance with one embodiment, a method of treating a subject in need of such treatment is provided. The method comprises administering a pharmaceutical composition comprising at least one compound of the present invention to a subject in need thereof. Compounds identified by the methods of the invention can be administered with known compounds or other medications as well.

The invention also encompasses the use of pharmaceutical compositions of an appropriate compound, and homologs, fragments, analogs, or derivatives thereof to practice the methods of the invention, the composition comprising at least one appropriate compound, and homolog, fragment, analog, or derivative thereof and a pharmaceutically-acceptable carrier.

The pharmaceutical compositions useful for practicing the invention may be administered to deliver a dose of between 1 ng/kg/day and 100 mg/kg/day.

The invention encompasses the preparation and use of pharmaceutical compositions comprising a compound useful for treatment of the diseases disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the invention is contemplated include, but are not limited to, humans and other primates, mammals including commercially relevant mammals such as cattle, pigs, horses, sheep, cats, and dogs, birds including commercially relevant birds such as chickens, ducks, geese, and turkeys. The invention is also contemplated for use in contraception for nuisance animals such as rodents.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically, dosages of the compound of the invention which may be administered to an animal, preferably a human, range in amount from 1 μg to about 100 g per kilogram of body weight of the animal. While the precise dosage administered will vary depending upon any number of factors, including but not limited to, the type of animal and type of disease state being treated, the age of the animal and the route of administration. Preferably, the dosage of the compound will vary from about 1 mg to about 10 g per kilogram of body weight of the animal More preferably, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even lees frequently, such as once every several months or even once a year or less. The frequency of the dose will be readily apparent to the skilled artisan and will depend upon any number of factors, such as, but not limited to, the type and severity of the condition or disease being treated, the type and age of the animal, etc.

Suitable preparations of vaccines include injectables, either as liquid solutions or suspensions, however, solid forms suitable for solution in, suspension in, liquid prior to injection, may also be prepared. The preparation may also be emulsified, or the polypeptides encapsulated in liposomes. The active immunogenic ingredients are often mixed with excipients which are pharmaceutically acceptable and compatible with the active ingredient. Suitable excipients are, for example, water saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants which enhance the effectiveness of the vaccine.

The invention is also directed to methods of administering the compounds of the invention to a subject. In one embodiment, the invention provides a method of treating a subject by administering compounds identified using the methods of the invention. Pharmaceutical compositions comprising the present compounds are administered to an individual in need thereof by any number of routes including, but not limited to, topical, oral, intravenous, intramuscular, intra arterial, intramedullary, intrathecal, intraventricular, transdermal, subcutaneous, intraperitoneal, intranasal, enteral, topical, sublingual, or rectal means.

In accordance with one embodiment, a method of treating and vaccinating a subject in need of such treatment is provided. The method comprises administering a pharmaceutical composition comprising at least one compound of the present invention to a subject in need thereof. Compounds identified by the methods of the invention can be administered with known compounds or other medications as well.

For oral administration, the active ingredient can be administered in solid dosage forms, such as capsules, tablets, and powders, or in liquid dosage forms, such as elixirs, syrups, and suspensions. Active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate, and the like. Examples of additional inactive ingredients that may be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

A variety of vaginal drug delivery systems is known in the art. Suitable systems include creams, foams, tablets, gels, liquid dosage forms, suppositories, and pessaries. Mucoadhesive gels and hydrogels, comprising weakly crosslinked polymers which are able to swell in contact with water and spread onto the surface of the mucosa, have been used for vaccination with peptides and proteins through the vaginal route previously. The present invention further provides for the use of microspheres for the vaginal delivery of peptide and protein drugs. More detailed specifications of vaginally administered dosage forms including excipients and actual methods of preparing said dosage forms are known, or will be apparent, to those skilled in this art. For example, Remington's Pharmaceutical Sciences (15th ed., Mack Publishing, Easton, Pa., 1980) is referred to.

The invention also includes a kit comprising the composition of the invention and an instructional material which describes adventitially administering the composition to a cell or a tissue of a mammal. In another embodiment, this kit comprises a (preferably sterile) solvent suitable for dissolving or suspending the composition of the invention prior to administering the compound to the mammal

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviation the diseases or disorders in a cell or a tissue of a mammal. The instructional material of the kit of the invention may, for example, be affixed to a container which contains the peptide of the invention or be shipped together with a container which contains the peptide. Alternatively, the instructional material may be shipped separately from the container with the intention that the instructional material and the compound be used cooperatively by the recipient.

### EXAMPLES

### Cell and Tissues

The present application uses a number of normal and cancerous tissues and cell lines. The cancer cell lines tested include: lung squamous carcinoma cells, myeloid leukemia cells, osteosarcoma cells, human cervical adenocarcinoma cells, adenocarcinoma of the colon, colon cancer, and breast cancer. The human cells used include: 2 lung squamous cell carcinoma cell lines—H520 and A549; 2 myeloid leukemia cell lines—HL60 and K562; 2 osteosarcoma cell lines—U2OS and 143B; HEK293—human kidney epithelial cells (tumorigenic in nude mice); HeLa—human cervical adenocarcinoma cells; SW480—human adenocarcinoma of the colon; DLD2—human colon cancer cell line; MCF7 (GSM715720)—human breast cancer cell line; BT474 (GSM715717)—human breast cancer cell line; HCC38 (GSM715718)—human breast cancer cell line; MDA-MB134 (GSM715695)—human breast cancer cell line; MB-MDA231—human breast cancer cell line; IMR90—normal human fibroblast cell line; GSM541796 undifferentiated human embryonic stem cells; GSM541797 differentiated human embryonic stem cells.

Tissue from five normal breast samples was used in some experiments.

### Analysis of the Small RNA Data

The data analyzed in this section were downloaded from either the GEO database (see the NCBI website) or NCBI SRA database at their website. We considered only those sets of high throughput sequencing data where the size of the small RNA was 14-36 bases. For each dataset we looked for the processed sequence along with its cloning frequency. In case of non-availability of this data, the raw data were used to generate the unique sequence and its cloning frequency. The adaptor sequences from the raw data were removed using “Cutadapt” (version 1.0) program. For clarity of data each figure in this manuscript has been provided with either a GEO or a SRA accession number of the library that was used to generate the figure.

Building and Mapping of Small RNA on “tRNAdb”

Information about the tRNA genes in each species was downloaded from the “Genomic tRNA database” (See the UCSC website) (Chan and Lowe 2009). For each tRNA gene the DNA sequences ranging from 100 bases upstream of the start of mature tRNA to 200 bases downstream of the end of mature tRNA were extracted from the same genome assembly on which the tRNA gene coordinates were built. A species-specific tRNA database called “tRNAdb” was built. To find the tRNA-related RNA sequences in each library, the small RNAs were mapped on the species-specific tRNAdb, using BLASTn (Altschul et al. 1997). In general we considered only those alignments where the query sequence (small RNA) was mapped to the subject sequence (tRNA) along 100% of its length. The blast output file was parsed to get information on the mapped position of small RNA on tRNA genes. We extract all map positions where the small RNA aligned from its first base to the last base with tRNA sequence allowing either one or no mismatch. Since “CCA” is added at the 3′ end of tRNA by tRNA nucleotidyltransferase during maturation of tRNA (Xiong and Steitz 2006), we allowed a special exception for the small RNA mapping to the 3′ ends of tRNAs in the tRNAdb allowing a terminal mismatch of <=3 bases. To remove any false positives, the small RNAs that mapped on to the “tRNAdb” were again searched against the whole genome using blast search excluding the tRNA loci. Only those small RNAs were qualified as tRFs that mapped exclusively on tRNAdb.

### Hierarchical Clustering and Heat Map

The small RNA libraries of six B-cell lines (2 naïve B-cells (MCL114 and MCL112), 2 plasma B-cells (U266 and h929) and 2 germinal center B-cells (L428 and L1236)), 2 cell lines derived from lung squamous cell carcinoma (H520 and A549), 4 primary breast cell lines, 2 stem cell (differentiated and undifferentiated), 2 myeloid leukemia (HL60 and K562), one peripheral blood mononuclear cell isolated from blood of normal person, two IMR90 fibroblast cell lines (young and senescent), two osteosarcoma cell lines (U205 and 143B) and five normal breast tissues were considered for find tissue specificity of tRF-1. The selection of small RNA libraries was based on (1) the availability of >1 library derived from cell-lines of the same tissue and (2) similarity in the protocols and platforms for small RNA isolation and sequencing. The small RNAs were mapped on “human tRNAdb” and the number of reads of individual tRF-1 was counted and normalized to RPM. The RPM value of some of the tRF-1 (e.g., Chr10.trna2.SerTGA; SEQ ID NO:68) was very high compared to other tRF-1 and hence to improve the interpretability or appearance of the graph the RPM value of each tRF-1 was log transformed and was used for hierarchical clustering. The hierarchical clustering and heat map were generated using hclust and heatmap.2 program available in the Bioconductor package.

### Example 1

### Results

Characterization of tRFs in Human Cell Lines

We analyzed high-throughput sequencing data of small RNA isolated from various human cell lines (Mayr and Bartel 2009). The 5′ and 3′ ends of each tRF were mapped on the corresponding tRNA gene. FIG. 1A shows the frequency of tRF 5′ and 3′ ends mapped on each base of the tRNA genes from HEK293 human cell lines. If the tRFs are a result of the random degradation of tRNA then the ends of the tRFs are expected to be equally distributed along the lengths of the tRNA genes. This is clearly not the case. Instead, the tRFs mainly originate from three specific regions: 5′ end (tRF-5), 3′ end (tRF-3), and 3′ trailer region (tRF-1) of tRNA genes. The frequency of sequencing of tRF-5, 3, and -1 in various human cell lines as indicated (FIG. 1B). tRF-1 always end with an RNA polymerase III (RNA pol III) transcription terminal signal (UUUUU, UUCUU, GUCUU or AUCUU) (Hagenbuchle et al. 1979; Koski and Clarkson 1982) indicating that this series of tRFs are generated by endonucleolytic cleavage of pre-tRNAs during maturation. As can be seen in FIG. 1A, tRF-5 is more abundant than tRF-3 or -1, both of which are identified at about the same frequency.

The three classes of tRFs are very similar to our previous report on tRFs (Lee et al. 2009). To determine if the observed patterns of tRFs in HEK293 can be extended to other cell lines we analyzed the high-throughput sequencing data of small RNA extracted from nine different human cell lines: HeLa, U205, 143B, A549, H520, SW480, DLD2, MCF7, and MB-MDA231 (Mayr and Bartel 2009). The pattern of tRFs was similar in all the analyzed cell lines despite their different origins (FIG. 1B & Supplementary FIG. 1).

When considered as a class, the observed lengths for tRF-5 peaked at 18, 22 and 32 bases, corresponding to the 3′ cleavage at +18 (tRF-5a), +22 to +24 (tRF-5b) and +30 to +32 (tRF-5c) (FIG. 2A). These cleavage sites are in the D loop, D stem, or the 5′ half of the anticodon stem (FIG. 2C). The lengths of tRF-3 peaked at 22 and 18 bases, corresponding to 5′ cleavage at +55 (tRF-3a) and +59 to +60 (tRF-3b), both of which are in the TψC loop (FIGS. 2A and C). Most of the tRF-1 fragments are 15-22 bases long. Even when we restricted the analysis to the most abundant tRF-5, -3 and -1 (>20 reads per million) we observed a similar length distribution of the fragments (FIG. 2B). A similar trend of length distribution was observed in all the other human cell lines indicating the conservation in length of tRFs (Supplementary FIG. 2). The specific length distribution of the tRFs indicates that the tRFs are not the random products of tRNA degradation. Interestingly the tRFs generated from an individual tRNA family or tRNA gene is even more specific in the length, corresponding to cleavage at one or a few specific bases (FIG. 2D). In further support of this specificity, the same cleavage sites were identified for these specific tRFs in other human cell lines (Supplementary FIG. 3).

tRFs are Present in Other Species

We next analyzed tRFs in the publicly available small RNA data of mice (Babiarz et al. 2008; Mayr and Bartel 2009), D. melanogaster (Ameres et al. 2010), C. elegans (de Lencastre et al. 2010), S. pombe (Barraud et al. 2011) and S. cerevisiae (Drinnenberg et al. 2011) (FIG. 3A-F). tRF-5 and tRF-3 are observed in all the species (FIG. 3G). However fewer tRF-1 were observed in Drosophila (˜500) and none in C. elegans or S. cerevisiae, though about 7,000 tRF-1 were detected in S. pombe. One explanation could be that the tRF-1 generated in some of these species were not in the selected size range (14-36 nucleotide) of small RNA that were subjected to cloning and sequencing. The length of a tRF-1 depends on the distance between the RNA polymerase III transcription termination site (UUUUU, UUCUU, GUCUU, or AUCUU) from the end of the tRNA. We therefore computationally extracted the predicted lengths of tRF-1 of each tRNA gene in the various species. The length distribution of the 3′ trailer sequence in various species ranged from a few bases to a few hundred bases (FIG. 3H). tRF-1 of 14-36 bases are ten-fold lower in C. elegans and S. cerevisiae compared to human and mouse, which could account for the absence of tRF-1 in the small RNA purified from these species. On the other hand, Drosophila has comparable numbers of tRF-1 in the correct size range, and yet yielded fewer tRF-1. S. pombe on the other hand yielded a large number of tRF-1 clones despite having fewer tRF-1 in the correct size range. Thus, some other factor besides the possible number of tRF-1 in the correct size range, such as expression level, helps determine how many tRF-1 are stable and identifiable in the data sets.

All tRNAs do not Produce Three tRFs, and not all tRFs are Equally Abundant

To determine if all tRNA genes produce all three types of tRFs and if they do, whether the tRFs are in comparable abundance, we selected those tRNA genes where a tRF-1 of at least 20 Reads Per Million (RPM) is present in HEK293 human cell line. In humans there are 207 predicted tRF-1 of 14-36 bases (FIG. 3H). Most of this tRF-1 are unique sequences and can be assigned to a specific tRNA gene. However, such attribution is not possible for tRF-5 and tRF-3 because the relevant parts of the mature tRNA show high sequence identity across >4-5 tRNA genes encoding the same anticodon tRNA. Hence for comparison we selected a specific tRNA gene that yielded a tRF-1 and studied the tRF-5 or -3 from the corresponding tRNA family. To determine the abundance of tRF-5 and tRF-3 for these specific tRNAs the total small RNAs were again mapped on to these selected tRNA genes.

The comparison of the sequencing frequency of these matched sets of tRFs is shown in FIG. 4. Not all the tRFs are detected for a given tRNA gene and family. For example, tRF-5-SerTGA or tRF-3-GlyTCC or -LeuAAG are selectively absent though tRF-1 were detected in all three cases.

When all three tRFs from a given tRNA gene or family are detected, their cloning frequencies are not similar. In most cases, the tRF-1 sequencing frequency is higher than that of the tRF-5 or tRF-3. For example, tRNA4-leuTAA produces a tRF-1 that is nearly 40-50 fold more abundant than the tRF-5 or -3 generated from the leuTAA tRNA family. One could also imagine that tRF-5 and -3 are released from a tRNA partially annealed to each other, and so should be in equimolar concentration. However, there is very little evidence in support of this, with many examples where a tRF-5 or -3 is 10 to 100 fold more abundant than its partner. The non-equivalence of the concentrations of tRF-5, -3 or -1 from a given tRNA gene (or family) further supports the hypothesis that tRFs are non-random, stable products derived from specific tRNAs and pre-tRNAs.

tRF-3 is Generated by a Cleavage Between A/U-A/U Bases

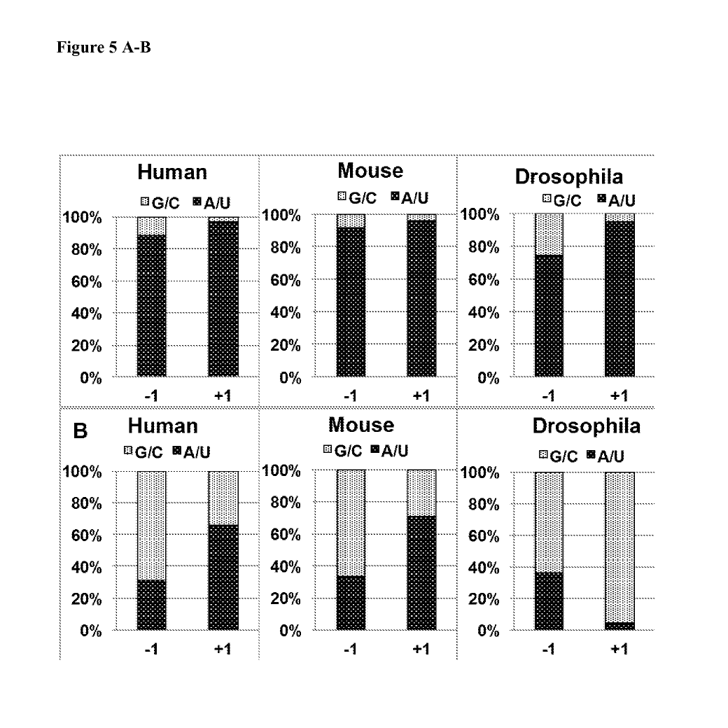

An “A” or “U” was present as the 5′ terminal base of the most abundant tRF-3 mapped on tRNAValCAC gene family. Indeed, “A” or “U” was noted as the 5′ terminal base of >95% of tRF-3 from humans, mice and flies (FIG. 5A). In addition an “A” or “U” was the immediate upstream base in the tRNA gene for >80% of tRF-3 in humans and mice, and >70% of tRF-3 in Drosophila. These results indicate that tRF-3 are most likely generated by an enzyme that preferentially cuts between A/U-A/U nucleotides in the T′PC loop.

A similar analysis of the 3′ ends of tRF-5 indicated that a weaker nucleotide bias also exists for tRF-5 (FIG. 5B). “G” or “C” was more abundant (>60-70%) compared to “A” or “U” at the 3′ end of tRF-5. However, the immediate downstream base was mostly “A” or “U” in human and mice. Interestingly, in Drosophila the base downstream from the tRF-5 cleavage site showed a strong bias for “G” or “C”. Therefore, the enzyme that cleaves tRNA to generate tRF-5 has a slight preference to cut between G/C-A/U bases in human and mice. However, in Drosophila, tRF-5 are most likely generated by an enzyme that preferentially cut between G/C-G/C nucleotides.

Processing of tRFs is Independent of Dicer or Drosha

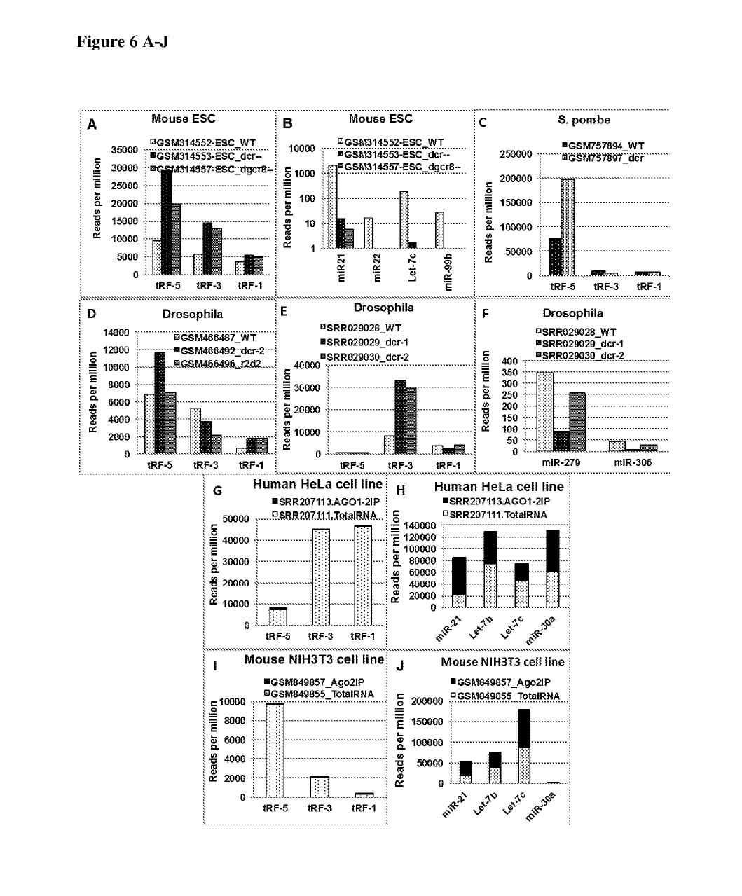

To study the role of Dicer protein in the generation of tRFs, we investigated the high throughput sequencing data of short RNAs from the wild type and Dicer mutants isolated under similar conditions from the same experiments. Such data were available for three species, i.e. Mouse (Babiarz et al. 2008), S. pombe (Barraud et al. 2011) and two data sets for Drosophila melanogaster (Zhou et al. 2009; Ghildiyal et al. 2010). Mutation of Dicer did not decrease the expression of all the three tRFs in mice (FIG. 6A), S. pombe (FIG. 6C) and D. melanogaster (FIG. 6D-E) in contrast to the nearly hundred-fold suppression of the cloning frequency of several microRNAs in mouse (FIG. 6B) and three- to twenty-fold suppression in Drosophila (FIG. 6F). Similar results were seen is mouse embryonic stem cells that were mutants for DGCR8 (an essential partner for the Drosha complex that cleaves pri-miRNA to generate pre-miRNA). Dicer-1 is involved in miRNA processing and Dicer-2 is a siRNA-processing enzyme in Drosophila. In addition to Dicer-2 the other double strand RNA binding protein R2D2 in fly is also involved in the biogenesis of siRNA. The mutant of R2D2 did not show any decrease in tRF expression as well. As a positive control, note that miRNA expression was significantly decreased in Dicer-1 mutant in D. melanogaster compared to wild type strain but not in the Dicer-2 mutant (FIG. 6F). The mutation in Dicer-1, Dicer-2, or R2D2 did not decrease the expression of tRF-5 and -1 either (FIG. 6D-E). Although tRF-3 was decreased to about 40% in the R2D2 mutant, in the context of all the other mutants, we conclude that the proteins involved in generating canonical miRNAs or siRNAs are dispensable for the generation of tRFs in mice, Drosophila and S. pombe. A more stringent question is whether the proteins involved in generating canonical miRNAs or siRNAs are utilized for generating any one specific tRF. We did not, however, find even one tRF that was significantly decreased in expression in the cells with Dicer-1 or Dicer-2 mutations relative to wild type cells.

tRFs are not Associated with Ago1/2

A strong bias for “A” or “U” at the 5′ end has been observed in many microRNAs and is reported to have a role in the loading of those short RNAs with Ago proteins. In addition, one report suggests that selected tRFs can associate with Ago proteins (Haussecker et al. 2010). We therefore examined the association of Ago 1/2 proteins with tRFs, particularly tRF-3.

We retrieved the sequencing data for total small RNAs from HeLa cells as well as of small RNAs immunoprecipitated with Ago1/2 protein isolated from the same cells (Valen et al. 2011). Similar data were also available for mouse NIH3T3 cells (Marcinowski et al. 2012). In both species, <2% of the tRFs were associated with Ago-1/2 protein (FIG. 6G-I). In contrast, 80% of mir-21 was associated with Ago 1/2 protein in the same experiment (FIG. 6H). Thus, although tRFs, particularly tRF-3 (Haussecker et al. 2010), can associate with Ago1 or 2, only a small minority of the tRFs may do so compared to the microRNAs. Even when we focused on the RPM of individual tRF there was no significant association of any tRF with Ago-1/2 protein. Even for highly abundant tRFs <1% of a given tRF was present in the Ago-1/2 immunoprecipitates compared to total RNA.

Cytoplasmic Vs. Nuclear Abundance of tRFs

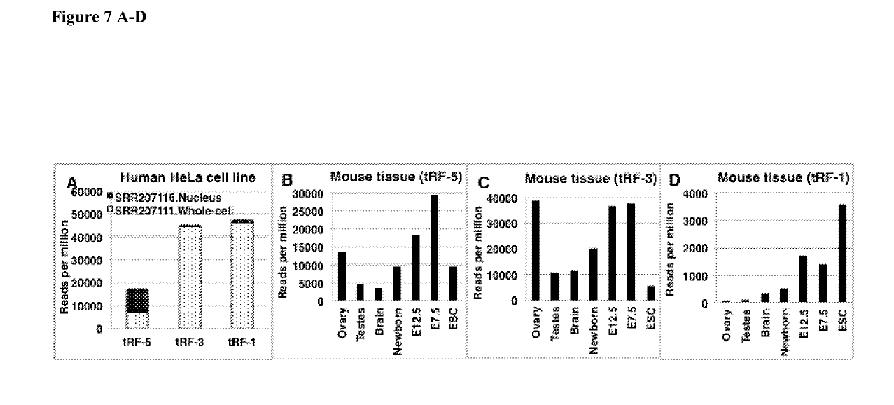

To determine the cytoplasmic and nuclear distribution of tRFs we analyzed the small RNA of 18-30 bases isolated separately from nuclei and whole cell fraction of HeLa cell lines (Valen et al. 2011) (FIG. 7A). The tRF-5 were equally present in the whole cell and nuclear fractions, suggesting that they may be exclusively present in the nucleus. tRF-3 and tRF-1 were much more abundant in the whole cell fraction compared to the nuclear fraction suggesting that both species are almost exclusively in the cytoplasm.

tRFs are Expressed in Normal Tissues

All the analyses of mammalian tRFs till now have been performed against RNA extracted from cell lines. To investigate if the tRFs are also expressed in normal mammalian tissues, we analyzed the small RNA data isolated from mouse ovary, testis and brain (Chiang et al. 2010). In addition, we also analyzed the small RNA isolated from mouse embryos and embryonic stem cells (Babiarz et al. 2008; Chiang et al. 2010). tRFs are present in all the tissues analyzed (FIG. 7B-D), but the tRF-5 and tRF-3 were more abundant in embryos and ovaries, and 2-5 fold less abundant in testis and brain. In contrast, the tRF-1 were less abundant in adult tissues compared to mouse embryo tissues and highly enriched in mouse embryonic stem cells. tRF-1 expression is markedly increased in malignant B cells

To investigate the expression of tRFs in normal and cancer cells we analyzed small RNAs (17-25 nt long) extracted from normal or malignant human B cells (Jima et al. 2010).

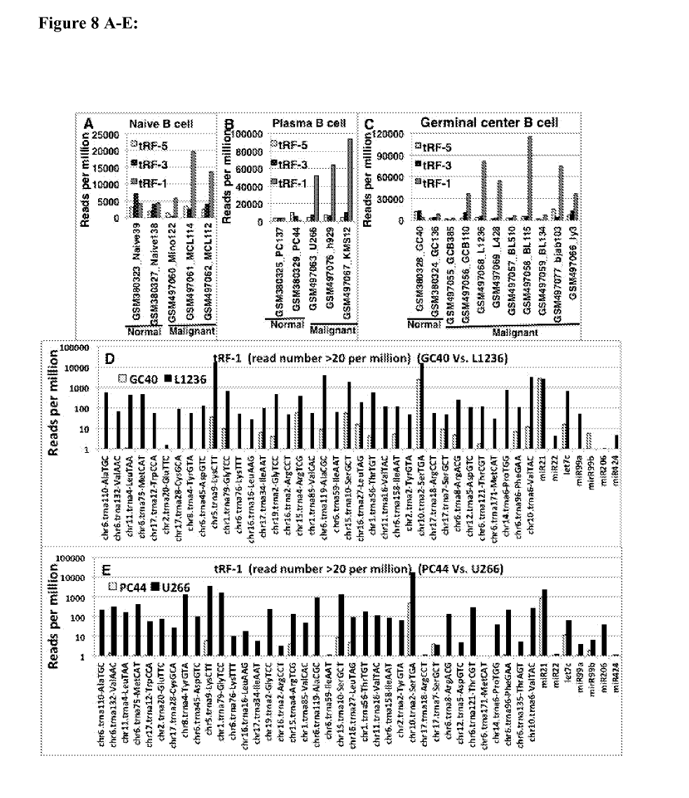

Small RNA was isolated from four subsets of B-cells (naive, germinal center, memory and plasma cell) from normal human subjects in two replicates (from two different individuals). Additionally, small RNAs were isolated from human B-cell derived tumors for each B-cell subset. The abundance of tRFs in normal as well as malignant B-cells in different subsets of B-cell is shown in FIG. 8A-C. tRF-1 was found to be more abundant in the malignant compared to normal in all the sub-sets of B-cells. In contrast, the abundance of tRF-5 and tRF-3 are not significantly different in normal and malignant B-cells.

To identify individual tRFs that are differentially expressed between the normal and malignant B-cells we extracted all those tRFs that were detected at >20 RPM either in normal or transformed B-cells (FIG. 8D, E). For many of the individual tRF-1s we observe a 100-1000× increase in abundance in the malignant B-cells compared to normal B-cells. However tRF-5 and tRF-3 do not exhibit a hundred fold induction in cancer B cells (Supplementary FIG. 4). If anything tRF-5 and -3 are often equal or less abundant in the malignant cells. Thus the increase in tRF-1 abundance is not simply a reflection of higher metabolism of tRNAs in the cancer cells.

Sequence Conservation of tRFs

The list of tRFs identified in this paper with a standard nomenclature will be curated as a database and will be made publicly available. The lists for the most abundant human tRF-5, 3, and -1 are shown in Supplemental Tables 1, 2 and 3, respectively. The Tables also indicate whether an individual tRF is conserved (maximum 2-base mis-match) between mice and humans and expressed in any of the mouse RNA libraries. We hope that this standardized nomenclature will facilitate comparison of tRFs between studies.

As tRF-5 and -3 are derived from mature tRNA, and tRNAs are conserved in sequence across species, we expected these tRFs to be conserved in sequence across species. In contrast, tRF-1 is derived from a non-functional part of the pre-tRNA, and so we were curious to see whether there was any sequence conservation of tRF-1 across species. Indeed, several identified tRF-1 (but not all) have sequence conservation from human to mouse (Table 1). In contrast, tRNA trailer sequences that did not yield tRF-1 in this study did not show such sequence conservation across species.

Expression of tRF-1 is Tissue-Specific.

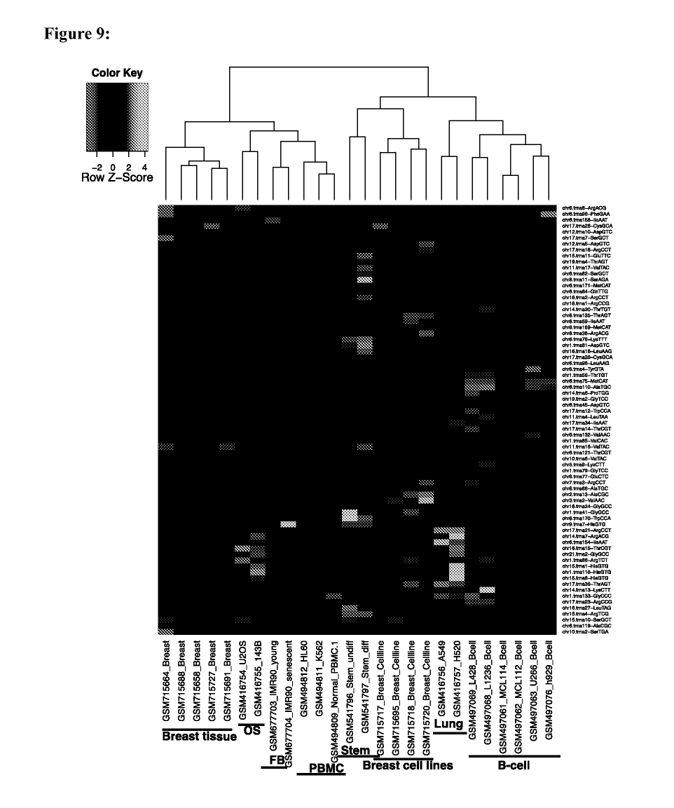

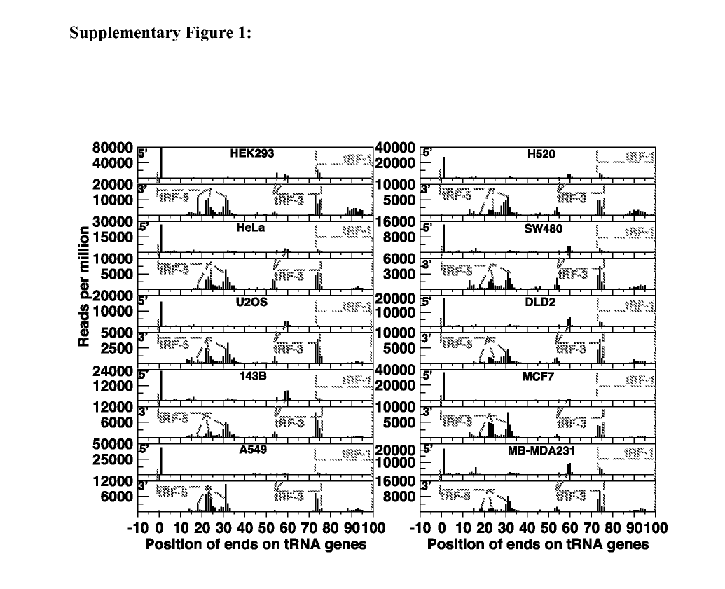

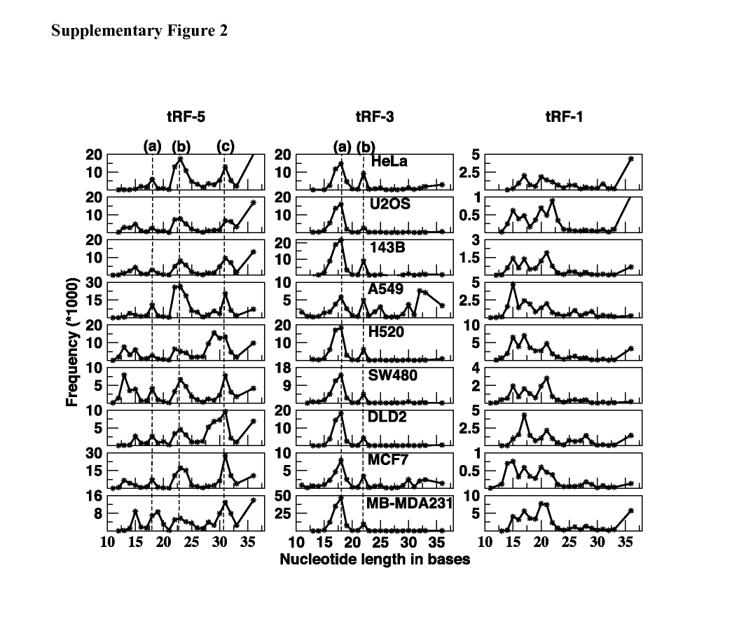

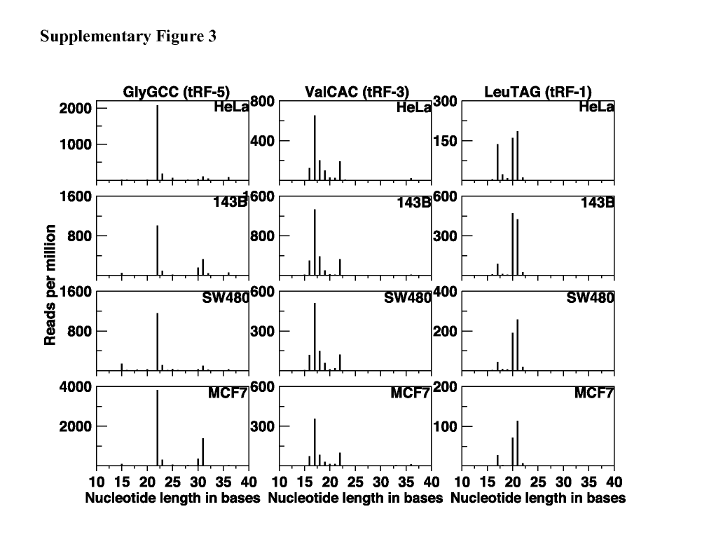

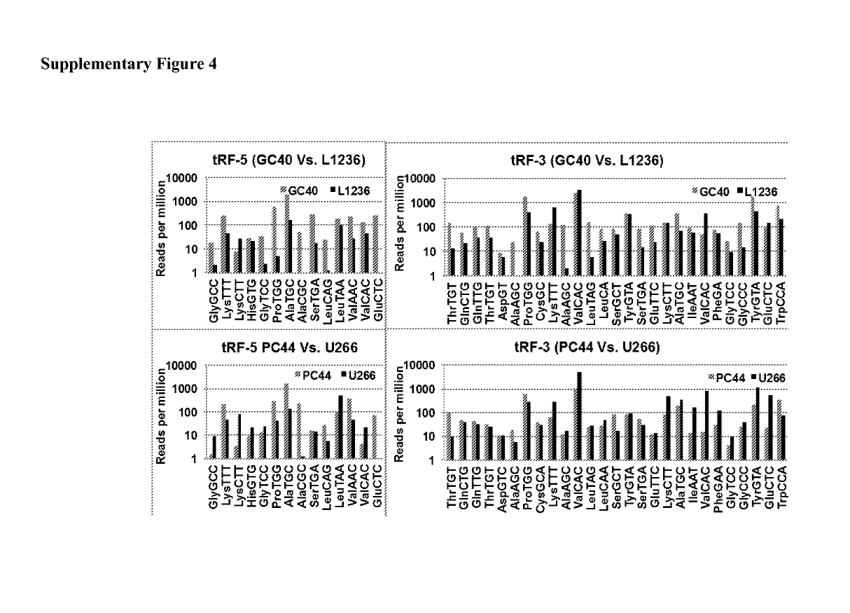

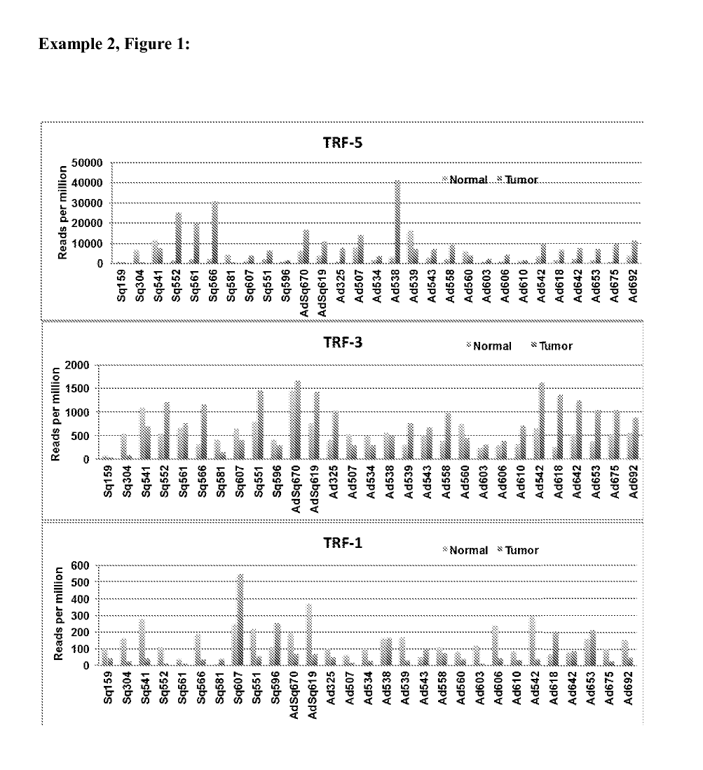

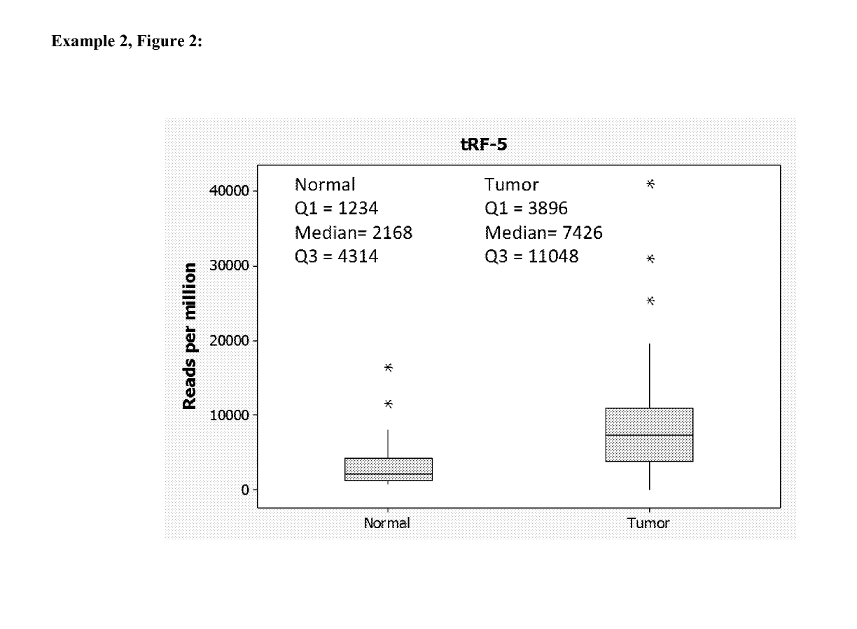

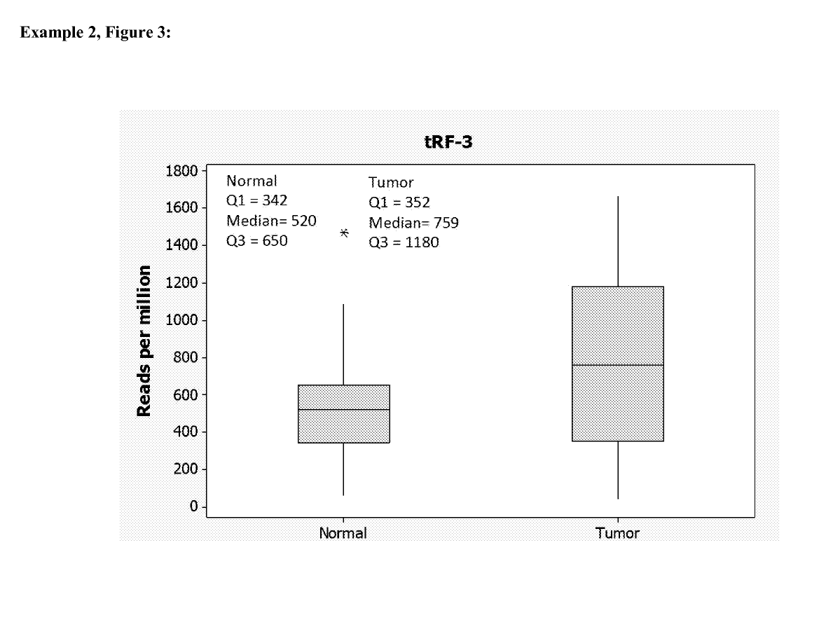

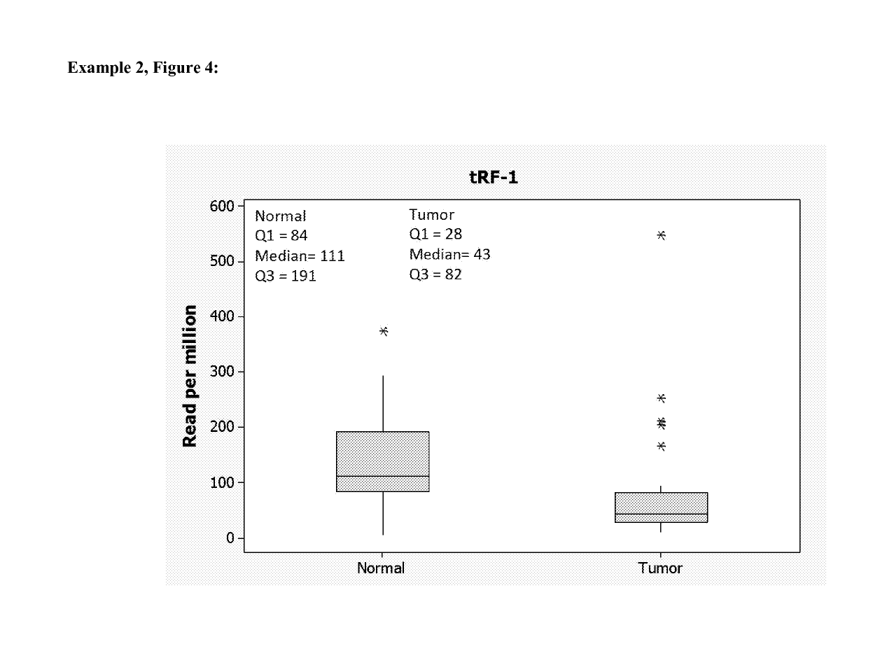

To investigate whether the expression of tRF-1 shows any specificity related to tissue of origin, we analyzed the small RNA libraries isolated from 6 B-cell lines (Jima et al., 2010) [2 naïve B-cells (MCL114 and MCL112), 2 plasma B-cells (U266 and h929) and 2 germinal center B-cells (L428 and L1236)], 2 cell lines derived from lung squamous cell carcinoma (H520 and A549) (Mayr and Bartel 2009), 4 primary breast cell lines (Farazi et al. 2011), 2 embryonic stem cell lines (differentiated and undifferentiated) (Bar et al. 2008), 2 myeloid leukemia (HL60 and K562) (Vaz et al. 2010), one peripheral blood mononuclear cell isolated from blood of normal person (Vaz et al. 2010), two IMR90 fibroblast cell lines (young and senescent) (Dhahbi et al. 2011), two osteosarcoma cell lines (U2OS and 143B) (Mayr and Bartel 2009) and five normal breast tissues (Farazi et al. 2011). The RPM value for each tRF-1 was log transformed. The hierarchical clustering and heat map of tRF-1 expression levels in various libraries is shown in FIG. 9. Cell lines generated from similar tissues clustered together in the heat map. It can be seen that the normal breast tissue libraries make a cluster that is separate from the breast cancer cell lines and this probably reflects the low epithelial content of normal breast tissue because we did not observe a difference in abundance of tRF-1 between normal breast tissue and breast cancer tissue (not shown). The clustering also distinguishes B-cell stages: naive (MCL114 and MCL112), plasma-cell (U266 and h929) and germinal center (L428 and L1236). Thus the clustering pattern indicates that expression of tRF-1 is influenced by tissue of origin and by stage of differentiation.

### Summary of Tables—

Table 1:

The alignment of conserved tRF-1 from Human, Chimp, Rhesus, Mouse, and Orangutan is given. When shown in color, the conserved residues are in red.

Supplementary Table 1:

List of tRF-5 that had abundance >20 reads per million in human cell lines. The amounts of each read are provided. * Name given in Lee at al. (Lee et al. 2009). + in the “M” column indicates that the sequence is conserved in mice and expressed in one of the mouse RNA libraries analyzed in this study. @ Representative tRNA gene name is according to GtRNAdb (Chan and Lowe 2009). Length is length in nucleotide residues for the fragment.

Supplementary Table 2:

List of tRF-3 that had abundance >20 reads per million in human cell lines. * Name given in Lee at al. (Lee et al. 2009). + in the “M” column indicates that the sequence is conserved in mice and expressed in one of the mouse RNA libraries analyzed in this study. @ Representative tRNA gene name is according to GtRNAdb (Chan and Lowe 2009).

Supplementary Table 3:

List of tRF-1 that had abundance >20 reads per million in human cell lines. * Name given in Lee at al. (Lee et al. 2009). + in the “M” column indicates that the sequence is conserved in mice and expressed in one of the mouse RNA libraries analyzed in this study. tRNA gene name is according to GtRNAdb (Chan and Lowe 2009).

TABLE 1

| Seq ID No. | | |
|---|---|---|
| | chr17.trna12-TrpCCA | |
| 100 | Human | ----AGGTTGGGTTTT |
| 101 | Chimp | ----AGGTTGGGTTTT |
| 102 | Rhesus | GGGGTGGTTGTGTTTT |
| 103 | Mouse | -----AGTTAGGTTTT |
| 104 | Orangutan | AACGAAGAATTGTTTT |
| | chr19.trna2-GlyTCC | |
| 105 | Rhesus | -GCGGGCCGACCTTTT |
| 106 | Orangutan | -GCGGGCCGACCTTTT |
| 107 | Mouse | -GCGTGCCCACGTTTT |
| 98 | Human | TGCGGTACCAC-TTTT |
| 108 | Chimp | TGCGATGTTAC-TTTT |
| | chr1.trna56-ThrTGT | |
| 109 | Human | CCTGTTGGC--TTACTTTT |
| 110 | Chimp | CCTGTTGGC--TTCCTTTT |
| 111 | Rhesus | CCTGTCGGC--TTACTTTT |
| 112 | Mouse | ----TAAGG--TTACTTTT |
| 113 | Orangutan | ----TCTGGAATTAATTTC |
| | chr2.trna2-TyrGTA | |
| 114 | Human | CTTCGTCTGTAA-TTTT |
| 115 | Chimp | CTTCGTCTGTAA-TTTT |
| 116 | Mouse | CTTCGTGCACTACTTTT |
| 117 | Rhesus | CTTCGTGTACCA-TTTT |
| 118 | Orangutan | CTTCGTGTATCA-TTTT |
| | chr6.trna45-AspGTC | |
| 119 | Human | ----GGCTTAAAC-TTTT |
| 120 | Orangutan | --AAGACCTAAGCCTTTT |
| 121 | Rhesus | --GAGACTTCAG--TTTT |
| 122 | Chimp | --GAGGCTTAAG--TTTT |
| 123 | Mouse | AAGATGGCTAAA--TTTT |
| | chr16.trna2-ArgCCT | |
| 124 | Chimp | AAGAAAGG-CTGAA-TTTT |
| 125 | Orangutan | AGGAAAGG-CTGA-GTTTT |
| 126 | Human | AAGAAAGG-CCGAA-TTTT |
| 127 | Mouse | -A-AAAGGACT-A--TTTT |
| 128 | Rhesus | ---AAAGT-GGCAAGTTTC |
| | chr6.trna158-IleAAT | |
| 129 | Human | ---CTTCCGT-GGGTTTGT |
| 130 | Chimp | ---CTTACGTAGGGTTTTT |
| 131 | Orangutan | -AGTGTTCGTTGCGCTTTT |
| 132 | Rhesus | GAGGGTGGTTTGTTGTTTT |
| 133 | Mouse | --GGGGAGTTT---GTTTT |
| | chr1.trna79-GlyTCC | |
| 134 | Rhesus | -GCGGGCCGACCTTTT |
| 135 | Orangutan | -GCGGGCCGACCTTTT |
| 136 | Mouse | -GCGTGCCCACGTTTT |
| 80 | Human | GCGGTACCAC-TTTT |
| 137 | Chimp | TGCGATGTTAC-TTTT |
| | chr10.trna6-ValTAC | |
| 75 | Human | ---TGGTGTGGTCTGTTG-TTTT |
| 138 | Chimp | ---TGGTGTGGTCTGTTG-TTTT |
| 139 | Mouse | -CGTGGTGTGCTA-GTTAATTTT |
| 140 | Orangutan | -CGGGGTGTTACATTGTG-TTTT |
| 141 | Rhesus | TGGTCGCGAGGCGGC-TC-TTTT |
| | chr15.trna4-AxgTCG | |
| 91 | Human | --AAGGGAGGTTATGATTAACTTTT |
| 142 | Chimp | --CAGGGAGGTTATGACTAACTTTT |
| 143 | Orangutan | --CAGCGAGGTGGTGAATAACTTTT |
| 144 | Mouse | ---------------CTTAACTTTT |
| 145 | Rhesus | AATGATGGTGTGATGACAAACTTTT |
| | chr11.trna16-ValTAC | |
| 146 | Rhesus | --TG-GTGAGGTCTAC---TATTTT |
| 147 | Mouse | CGTGGTGTGCTAGTTAATTTT |
| 148 | Human | --CGGCGTGAT-TCATACC---TTTT |
| 149 | Chimp | --CGGCGTGAT-TCACACC---TTTT |
| 150 | Orangutan | -CGGGGTG-T-T-ACATTGTGTTTT |
| | chr10.trna2-SerTGA | |
| 151 | Orangutan | GAAGCGGGTGCTTACA-TTTT |
| 152 | Mouse | GAAGCGGGTGCTT-CACTTTT |
| 68 | Human | GAAGCGGGTGCTCTTA-TTTT |
| 153 | Chimp | GAAGCAGGTGCTTGTA-TTTT |
| 154 | Rhesus | GAAGCAGGTGCTTCTG TCTT |

SUPPLEMENTARY TABLE 1

| SEQ ID | | | | | tRF-5 |
|---|---|---|---|---|---|
| NO: | tRNA gene name @ | tRF-5 sequence | Length | M | name |
| 1 | chr17.trna42-LeuTAG | GGTAGCGTGGCCGAGC | 16 | | 5003* |
| 2 | chr6.trna41-SerACT | GGCCGGTTAGCTCAG | 15 | | 5007* |
| 3 | chr6.trna8-ArgACG | GGGCCAGTGGCGCAATGG | 18 | | 5018* |
| 4 | chr15.trna11-GluTTC | TCCCACATGGTCTAGCGGTTAGG | 23 | + | 5021* |
| 5 | chr7.trna9-TyrGTA | GGGGGTATAGCTC | 13 | | 5035* |
| 6 | chr6.trna70-AlaCGC | GGGGGTGTAGCTCAGTGGTAGAGCGCGTGC | 30 | + | 5037 |
| 7 | chr17.trna23-ArgCCG | GACCCAGTGGCCTA | 14 | | 5038 |
| 8 | chr12.trna5-AspGTC | TCCTCGTTAGTATAGTGG | 18 | | 5039 |
| 9 | chr6.trna48-AspGTC | TCCTCGTTAGTATAGTGGTGAGT | 23 | + | 5040 |
| 10 | chr4.trna3-CysGCA | GGGGGTATAGCTCAGT | 32 | + | 5041 |
| | | GGTAGAGCATTTGACT | | | |
| 11 | chr14.trna8-CysGCA | GGGGTATAGCTCAGGGGAGAGCATTTGACT | 30 | + | 5042 |
| 12 | chr1.trna27-GlnCTG | GGTTCCATGGTGTA | 14 | | 5043 |
| 13 | chr6.trna87-GluCTC | TCCCTGGTGGTCTAGTGGTTAGG | 23 | + | 5044 |
| 14 | chr2.trna20-GluTTC | TCCCATATGGTCTAGCGGTTAGG | 23 | + | 5045 |
| 15 | chr1.trna64-GluTTC | TCCCTGTGGTCTAGTGGTTAGGA | 23 | + | 5046 |
| 16 | chr2.trna27-GlyCCC | GCGCCGCTGGTGTAGTGGTATCATGCAAGA | 30 | + | 5047 |
| 17 | Chr21.trna2-GlyGCC | GCATGGGTGGTTCAGTGGTAGA | 22 | + | 5048 |
| 18 | chr6.trna128-GlyGCC | GCATTGGTGGTTCAGTGGTAGA | 22 | + | 5049 |
| 19 | chr1.trna79-GlyTCC | GCGTTGGTGGTATAGTGGTGAGC | 23 | + | 5050 |
| 20 | chr6.trna7-LeuCAG | GTCAGGATGGCCGAGCGGTCTAA | 23 | + | 5051 |
| 21 | chr4.trna2-LeuTAA | GTTAAGATGGCAGAGCCCGGTAATCGCATA | 30 | | 5052 |
| 22 | chrX.trna2-LeuTAA | GTTAAGATGGCAGAGCCCG | 19 | | 5053 |
| 23 | chr6.trna13-LysCTT | GCCCGGCTAGCTCAGTCGGTAGAGCATGAGA | 31 | + | 5054 |
| 24 | chr15.trna2-LysCTT | GCCCGGCTAGCTCAGT | 32 | + | 5055 |
| | | CGGTAGAGCATGGGAC | | | |
| 25 | chr6.trna76-LysTTT | GCCCGGATAGCTCAGTCGGTAGAGCATCAGA | 31 | + | 5056 |
| 26 | chr5.trna14-ProTGG | GGCTCGTTGGTCTAGGGGTATGATTCTCGC | 30 | + | 5057 |
| 27 | chr11.trna12-ProTGG | GGCTCGTTGGTCTAGGG | 17 | | 5058 |
| 28 | chr14.trna3-ProTGG | GGCTCGTTGGTCTAG | 15 | | 5059 |
| 29 | chr6.trna51-SerTGA | GTAGTCGTGGCCGAGTGGTTAAG | 23 | + | 5060 |
| 30 | chr8.trna5-TyrGTA | CCTTCGATAGCTCAG | 15 | | 5061 |
| 31 | chr5.trna15-ValAAC | GTTTCCGTAGTGTAGTGGTCATCACGTTCGC | 31 | + | 5062 |
| 32 | chr6.trna152-ValCAC | GCTTCTGTAGTGTAGTGGTTATCACGTTCGC | 31 | + | 5063 |
| 33 | chr6.trna9-ValCAC | GTTTCCGTAGTGTAGTGGTTATCACGTTCGC | 31 | + | 5064 |
| 34 | chrX.trna4-ValTAC | GGTTCCATAGTGTAGTGGTTATCACGTCTGC | 31 | + | 5065 |

SUPPLEMENTARY TABLE 2

| SEQ ID | tRNA gene | tRF-3 | | | tRF-3 |
|---|---|---|---|---|---|
| NO: | name @ | sequence | Length | M | name |
| 35 | chr16.trna27- | ATCCCACCG | 18 | | 3001* |
| | LeuTAG | CTGCCACCA | | | |
| 36 | chr6.trna65- | TCCCCGGCA | 18 | | 3003* |
| | AlaAGC | CCTCCACCA | | | |
| 37 | chr6.trna66- | TCCCCGGCA | 18 | | 3004* |
| | AlaTGC | TCTCCACCA | | | |
| 38 | chr21.trna2- | TCGATTCCCGG | 22 | + | 3006* |
| | GlyGCC | CCCATGCACCA | | | |
| 39 | chr19.trna2- | TCGATTCCCGG | 22 | + | 3007* |
| | GlyTCC | CCAACGCACCA | | | |
| 40 | chr6.trna83- | ACCCCACTC | 18 | | 3009* |
| | LeuTAA | CTGGTACCA | | | |
| 41 | chr5.trna6- | ACCGGGCGG | 17 | | 3011* |
| | ValCAC | AAACACCA | | | |
| 42 | chr1.trna26- | CCCACCCAG | 17 | | 3013* |
| | AsnGTT | GGACGCCA | | | |
| 43 | chr6.trna76- | TCCCTGTTC | 17 | | 3015* |
| | LysTTT | GGGCGCCA | | | |
| 44 | chr6.trna7- | ATCCCACTC | 18 | | 3016* |
| | LeuCAG | CTGACACCA | | | |
| 45 | chr17.trna5- | TCGATTCCCGG | 22 | + | 3018* |
| | GlyGCC | CCAATGCACCA | | | |
| 46 | chr16.trna8- | ATCCCGGAC | 18 | | 3019* |
| | ProTGG | GAGCCCCCA | | | |
| 47 | chr14.trna2- | ATCCCACCA | 18 | | 3022* |
| | LeuTAG | CTGCCACCA | | | |
| 48 | chr6.trna74- | ATCCCACTT | 18 | | 3026* |
| | LeuCAA | CTGACACCA | | | |
| 49 | chr12.trna11- | CCCGGGTTT | 17 | | 3034* |
| | PheGAA | CGGCACCA | | | |
| 50 | chr4.trna3- | TCCGGGTGC | 17 | | 3039* |
| | CysGCA | CCCCTCCA | | | |
| 51 | chr9.trna7- | TCCGAGTCA | 17 | | 3041* |
| | HisGTG | CGGCACCA | | | |
| 52 | chr6.trna80- | TCCCCGTAC | 18 | | 3052* |
| | IleAAT | GGGCCACCA | | | |
| 53 | chr1.trna44- | TCGATTCCCCG | 22 | + | 3053* |
| | AspGTC | ACGGGGAGCCA | | | |
| 54 | chr2.trna2- | TCCGGCTCG | 17 | | 3057* |
| | TyrGTA | AAGGACCA | | | |
| 55 | chr1.trna56- | TCTCGCTGG | 17 | | 3066* |
| | ThrTGT | GGCCTCCA | | | |
| 56 | chr11.trna16- | TCGAGCCCCAG | 22 | + | 3070* |
| | ValTAC | TGGAACCACCA | | | |
| 57 | chr6.trna99- | TCTCGGTGG | 17 | | 3072* |
| | GlnCTG | AACCTCCA | | | |
| 58 | chr17.trna16- | TCTCGGTGG | 17 | | 3075* |
| | GlnTTG | GACCTCCA | | | |
| 59 | chr6.trna108- | TCCCCAGTA | 18 | | 3078 |
| | AlaAGC | CCTCCACCA | | | |
| 60 | chr6.trna48- | GGTTCGATTCCCC | 25 | + | 3079 |
| | AspGTC | GACGGGGAGCCA | | | |
| 61 | chr13.trna4- | TCGATTCCCGG | 22 | + | 3080 |
| | GluCTC | TCAGGGAACCA | | | |
| 62 | chr2.trna18- | TCGTTTCCCGG | 22 | + | 3081 |
| | GluCTC | TCAGGGAACCA | | | |
| 63 | chr13.trna3- | TCGACTCCCGG | 22 | + | 3082 |
| | GluTTC | TGTGGGAACCA | | | |
| 64 | chr14.trna13- | TCGAGCCCCAC | 22 | + | 3083 |
| | LysCTT | GTTGGGCGCCA | | | |
| 65 | chr16.trna20- | TCGAGCCTCAG | 22 | + | 3084 |
| | MetCAT | AGAGGGCACCA | | | |
| 66 | chr6.trna51- | ATCCTGCCGA | 18 | | 3085 |
| | SerTGA | CTACGCCA | | | |
| 67 | chr6.trna16- | TCCGGCTCG | 17 | | 3086 |
| | TyrGTA | GAGGACCA | | | |

SUPPLEMENTARY TABLE 3

| SEQ ID | | | | | tRF-1 |
|---|---|---|---|---|---|
| NO: | tRNA gene name | TRF-1 sequence | Length | M | name |
| 68 | chr10.trna2-SerTGA | GAAGCGGGTGCTCTTATTTT | 20 | + | 1001* |
| 69 | chrl7.trna7-SerGCT | GCTAAGGAAGTCCTGTGCTCAGTTTT | 26 | + | 1003* |

| | | | | | |
|---|---|---|---|---|---|
| 70 | chr12.trna5-AspGTC | GTGTGTAGCTGCACTTTT | 18 | | 1004* |
| 71 | chr15.trna10-SerGCT | ATGTGGTGGCTTACTTT | 17 | | 1005* |
| 72 | chr6.trna8-ArgACG | GTGTAAGCAGGGTCGTTTT | 19 | | 1006* |
| 73 | chr6.trna64-GlnTTG | TTCAAAGGTGAACGTTT | 17 | | 1007* |
| 74 | chr6.trna121-ThrCGT | TAGGGTGTGCGTGTTTTT | 18 | | 1008* |
| 75 | chr10.trna6-ValTAC | TGGTGTGGTCTGTTGTTTT | 17 | | 1010* |
| 76 | chr21.trna2-GlyGCC | GCACGAAAATGTGTTTT | 17 | | 1012* |
| 77 | chr6.trna119-AlaCGC | GGCGATCACGTAGATTTT | 18 | | 1013* |
| 78 | chr17.trna26-CysGCA | TGTGCTCCGGAGTTACCTCGTTT | 23 | + | 1015* |
| 79 | chr6.trna96-PheGAA | GAGAGCGCTCGGTTTTT | 17 | | 1020* |
| 80 | chr1.trna79-GlyTCC | GCGGGCGGACCTTTT | 15 | | 1023 |
| 81 | chr5.trna9-LysCTT | GCAACTGGTCGTTTT | 15 | | 1024 |
| 82 | chr6.trna154-IleAAT | GAGGGTTCTCACCTTCTCTCTCCGATTT | 28 | + | 1025 |
| 83 | chr6.trna158-IleAAT | TTCCGTGGGTTTGTTTT | 17 | | 1026 |
| 84 | chr6.trna171-MetCAT | ATGGCCGCATATATTT | 16 | | 1027 |
| 85 | chr6.trna45-AspGTC | GAGGCTTAAACTTTT | 15 | | 1028 |
| 86 | chr6.trna66-AlaTGC | ATAGGTATTAAGGTTTT | 17 | | 1029 |
| 87 | chr8.trna11-SerAGA | GGAATGTCAGCTTTT | 15 | | 1030 |
| 88 | chr8.trna4-TyrGTA | ACAAGTGCGGTTTTTT | 16 | | 1031 |
| 89 | chr11.trna4-LeuTAA | AAGAGGAGTTGTTTT | 15 | | 1032 |
| 90 | chr14.trna7-ArgACG | GTGGGGTGCCTCACAGCTTCGCTGCGTGAGC | 36 | | 1033 |
| | | ATTTT | | | |
| 91 | chr15.trna4-ArgTCG | AAGGGAGGTTATGATTAACTTTT | 22 | + | 1034 |
| 92 | chr16.trna15-ThrCGT | GATATCCAACCTTCGGCTATAGGGTGGAGAC | 36 | + | 1035 |
| | | TTTTT | | | |
| 93 | chr16.trna16-LeuAAG | GGGTTGCTGTCTTTT | 15 | | 1036 |
| 94 | chr16.trna27-LeuTAG | ACCTCAGAAGGTCTCACTTT | 20 | + | 1037 |
| 95 | chr17.trna18-ArgCCT | AGGTGAAAGTTCCTTT | 16 | + | 1038 |
| 96 | chr17.trna21-ArgCCT | TCGAGAGGGGCTGTGCTCGCAAGGTTTCTTT | 31 | + | 1039 |
| 97 | chr17.trna34-IleAAT | GTGGGTGGCTTTTTT | 15 | | 1040 |
| 98 | chr19.trna2-GlyTCC | TGCGGTACCACTTTT | 15 | | 1041 |
| 99 | chr19.trna4-ThrAGT | AACCGAGCGTCCAAGCTCTTTCCATTTT | 28 | + | 1042 |

### Example 2

### Example 2 Discloses the Results of Further Experiments on Cancer, Emphasizing Lung Cancer

It can be seen in the bar graphs of the three panels of Example 2, FIG. 1 that tRF-5 (Example 2, FIG. 1A) and tRF-3 (Example 2, FIG. 1B) are increased and tRF-1 (Example 2, FIG. 1C) decreased in several human lung carcinomas compared to normal adjoining lung. The abundance of tRFs in normal lung tissue and carcinoma (expressed as reads of tRFs/million reads of short RNAs) is shown. The subset of tRF-5 and -3 are 10-20 fold higher in several tumors compared to normal. Ad=Adenocarcinoma; Sq=Squamous Cell carcinoma. It should be noted that tRF-1 is not increased in lung cancers.

It can be seen in the graph of Example 2, FIG. 2 that tRF-5 abundance is increased in human lung carcinomas compared to normal adjoining lung. The amount of tRF-5s in normal lung tissue and carcinoma (expressed as number of reads of tRF-5s/million reads of short RNAs) is shown. All tRF-5s are considered together. Box and whiskers plot shows the median and interquartile range for the data. Asterisks indicate outliers. The difference in expression levels is statistically significant (p-value 0.0019) which was calculated by paired t-test.

It can be seen in the bar graph of Example 2, FIG. 3 that tRF-3 abundance is increased in human lung carcinomas compared to normal adjoining lung. The abundance of tRF-3s in normal lung tissue and carcinoma (expressed as number of reads of tRF-3s/million reads of short RNAs) is shown. All tRF-3s are considered together. Box and whiskers plot shows the median and interquartile range for the data. Asterisks indicate outliers. The difference in expression levels is statistically significant (p-value 0.0134) which was calculated by paired t-test.

It can be seen in the final bar graph of Example 2 (Example 2, FIG. 4) that tRF-1 abundance is decreased in human lung carcinomas compared to normal adjoining lung. The abundance of tRF-1 s in normal lung tissue and carcinoma (expressed as number of reads of tRF-1s/million reads of short RNAs) is shown. All tRF-1 s are considered together. Box and whiskers plot shows the median and interquartile range for the data. Asterisks indicate outliers. The difference in expression levels is statistically significant (p-value 0.0211) which was calculated by paired t-test.

### DISCUSSION

Referring back to the questions posed in the introduction, our analysis showed that the tRFs are present in all human cell lines examined and are present in mice, Drosophila, C. elegans, S. pombe, and S. cerevisiae. Such wide-spread occurrence suggests that they are probably ubiquitously present in eukaryotes. tRF-1 may not be present in some organisms such as C. elegans or S. cerevisiae. Alternatively, they may be present but much shorter than the size range usually examined in short RNA sequencing studies.

Our analysis confirms our previous observation of the three types of tRFs, tRF-5, -3 and -1, with the added qualification that subsets like tRF-5a, -5b, -5c and tRF-3a and -3b may be distinguished by characteristic lengths. However, new major types of tRFs derived from other parts of the tRNA or pre-tRNA were not evident. The non-random mapping of tRF ends along the length of tRNAs, generation of tRFs from a few specific cleavage sites in a given tRNA and the conservation of tRFs across various cell lines and tissue samples within a species strongly suggest that tRFs are not random degradation products of tRNA.

Sequence analysis of tRNA genes suggests that >1000 different tRFs are possible in humans. Yet only a small fraction of these is actually observed. For example of the 207 1-series tRFs theoretically in the 14-36 base length range in humans we observed only 10-15% of the predicted tRF-1s in the small RNAs extracted and sequenced from various cell lines. Similarly, not all possible tRFs from a given tRNA gene or gene family are seen in a given cell, and even if they are seen, they are not present in equivalent concentrations. This, too, suggests that specific subsets of tRFs are generated or stabilized in cells.

The generation of tRFs is not dependent on the canonical miRNA processing machinery suggesting that tRFs are generated by a yet to be identified pathway. We had shown previously that at least one tRF-1 (SerTGA) (SEQ ID NO:68; named tRF-1 1001) was substantially suppressed when RNAseZ (or ELAC1), known to release the 3′ trailer sequence of pre-tRNA, is knocked down. Li et al. showed that Angiogenin, RNAseA, or RNAseI can cleave mature tRNA to release a fragment similar to tRF-3 (Li et al., 2012). Whether these enzymes actually generate tRF-3 in vivo is not currently known. The enzyme(s) that generates tRF-5 is unknown. Although tRFs have been reported to associate with Ago-1 and -2 proteins, our results suggest that this is more the exception than the rule. tRFs have also been shown to be associated with Ago-3, Ago-4, and PIWI proteins. Since we did not have access to high quality short RNA sequencing data from the corresponding immunoprecipitates, we could not determine whether these associations involve the majority of the tRF in a cell, or only a small minority fraction. Overall these results are consistent with the suggestion that the functions of tRFs are unlikely to be similar to that of microRNAs.

Li et al. (Li et al. 2012), published a paper that analyzed tRFs in HEK293 cells and mouse embryonic stem cells. Our bioinformatics results regarding the specific presence of tRF-5 and tRF-3 and the lack of requirement of Dicer or DGCR8 in the generation of mouse tRFs are in agreement. However they did not explore the tRF-1. Experimental data in that paper suggested that some tRF-3 can associate in a functional complex with Ago-2. We did not find much association of tRFs with Ago-2. However, tRFs may function by associating with other Ago proteins, particularly Ago-1, -3, and -4.

The sites of generation of the three classes of tRFs are unknown. The abundance of tRF-1 in the cytoplasm compared to the nucleus, confirmed the subcellular distribution of tRF-1001 (SEQ ID NO:68) derived from SerTGA (Lee et al. 2009). We showed that the corresponding pre-tRNA was also present mostly in the cytoplasm, so that it is possible that a select pool of pre-tRNA is exported out of the nucleus to give rise to tRF-1 in the cytoplasm (Lee et al. 2009). However, we cannot rule out that many of the tRF-1 may be generated from conventional pre-tRNA processing in the nucleus and are transported to the cytoplasm by an active mechanism. The cytoplasmic location of tRF-3 is probably due to cleavage of mature tRNA in the cytoplasm. In mammals, mature tRNAs are exported to the cytoplasm with the help of nuclear export receptor for tRNA (exportin-t in Xenopus) and this export requires the mature 5′ and 3′ end of tRNA, including the added CCA (Kutay et al. 1998). Although tRF-3 almost always ends with CCA, it does not have the 5′ end of the tRNA and so probably cannot be exported using the same mechanisms that export the mature tRNA. tRF-5, on the other hand, could be generated in the cytoplasm from exported mature tRNA, and then imported to the nucleus by active mechanisms or could be generated from mature tRNA in the nucleus and retained in the nucleus by specific proteins.

The greater abundance of tRF-1 in mouse embryos, embryonic stem cells and a variety of cell-lines, compared to adult mouse tissues, may indicate that tRF-1 are associated with cell proliferation. However, the low abundance in testis, known for its high rate of cell proliferation, runs counter to this hypothesis.

The absence of tRF-1 in adult tissues and its high abundance in malignant B cells is highly interesting. While that may also suggest that tRF-1 expression is correlated with cell-proliferation, this is by no means clear. In other cancer-normal comparisons (e.g. breast and cervix tissues) we failed to detect a stimulation of total tRF-1 abundance (data not shown). Comparison of the heat map of tRF-1 expression (FIG. 9) clearly distinguishes breast cancer from breast cancer cell lines but this probably only a reflection of the low epithelial content of breast tissue. At the very least, tRF-1 could serve as a biomarker for B cell cancers. If tRFs are released into the bloodstream, and, if they are stabilized by associated proteins or lipids, as reported for microRNAs, the levels of circulating tRFs detected in the plasma could be a marker for detecting certain types of cancer.

While the high abundance of specific lengths and types of tRFs indicates that they are not random byproducts of tRNA generation or turnover, we still cannot be certain that all tRFs will be functionally important. We showed that knockdown of one tRF (tRF-1001 from SerTGA) suppressed cell proliferation and increased the population of cells in the G₂ phase of the cell cycle, suggesting that this tRF is required for optimal passage through G₂ to mitosis (Lee et al. 2009). Certain tRF-3 have been isolated complexed with Ago-2 and can promote the cleavage of a matching target in vitro (Li et al. 2012). Based on this, and sequence similarity, a few tRF-3 have been suggested to suppress human endogenous retrovirus based repeat elements, or even HIV infection. The sequence conservation of tRF-1 across several species, and the specific sequence requirements of tRF-1001 from SerTGA (Lee et al. 2009), also suggest that tRFs may have a function based on their sequences. However, in the absence of genetic evidence, we cannot yet conclude whether many of the tRFs identified in this study have biological functions. Despite this, multiple groups have begun studying tRFs, so that our comprehensive list of identified tRFs and the suggested nomenclature will facilitate comparison of results between multiple groups and elucidate the biological functions of tRFs.

The disclosures of each and every patent, patent application, and publication cited herein are hereby incorporated by reference herein in their entirety.

Headings are included herein for reference and to aid in locating certain sections. These headings are not intended to limit the scope of the concepts described therein under, and these concepts may have applicability in other sections throughout the entire specification.

While this invention has been disclosed with reference to specific embodiments, it is apparent that other embodiments and variations of this invention may be devised by others skilled in the art without departing from the true spirit and scope of the invention.

### BIBLIOGRAPHY

1. Altschul S F, Madden T L, Schaffer A A, Zhang J, Zhang Z, Miller W, Lipman D J. 1997. Gapped BLAST and PSI-BLAST: a new generation of protein database search programs. Nucleic Acids Res 25(17): 3389-3402.
2. Ameres S L, Horwich M D, Hung J H, Xu J, Ghildiyal M, Weng Z, Zamore P D. 2010. Target RNA-directed trimming and tailing of small silencing RNAs. Science 328(5985): 1534-1539.
3. Aravin A A, Lagos-Quintana M, Yalcin A, Zavolan M, Marks D, Snyder B, Gaasterland T, Meyer J, Tuschl T. 2003. The small RNA profile during Drosophila melanogaster development. Dev Cell 5(2): 337-350.
4. Aravin A A, Naumova N M, Tulin A V, Vagin V V, Rozovsky Y M, Gvozdev V A. 2001. Double-stranded RNA-mediated silencing of genomic tandem repeats and transposable elements in the D. melanogaster germline. Curr Biol 11(13): 1017-1027.
5. Babiarz J E, Ruby J G, Wang Y, Bartel D P, Blelloch R. 2008. Mouse E S cells express endogenous shRNAs, siRNAs, and other Microprocessor-independent, Dicer-dependent small RNAs. Genes Dev 22(20): 2773-2785.
6. Bar M, Wyman S K, Fritz B R, Qi J L, Garg K S, Parkin R K, Kroh E M, Bendoraite A, Mitchell P S, Nelson A M et al. 2008. MicroRNA Discovery and Profiling in Human Embryonic Stem Cells by Deep Sequencing of Small RNA Libraries. Stem Cells 26(10): 2496-2505.
7. Barraud P, Emmerth S, Shimada Y, Hotz H R, Allain F H, Buhler M. 2011. An extended dsRBD with a novel zinc-binding motif mediates nuclear retention of fission yeast Dicer. Embo J 30(20): 4223-4235.
8. Bartel D P. 2004. MicroRNAs: genomics, biogenesis, mechanism, and function. Cell 116(2): 281-297.
9. Brennecke J, Aravin A A, Stark A, Dus M, Kellis M, Sachidanandam R, Hannon G J. 2007. Discrete small RNA-generating loci as master regulators of transposon activity in Drosophila. Cell 128(6): 1089-1103.
10. Buhler M, Spies N, Bartel D P, Moazed D. 2008. TRAMP-mediated RNA surveillance prevents spurious entry of RNAs into the Schizosaccharomyces pombe siRNA pathway. Nat Struct Mol Biol 15(10): 1015-1023.
11. Chan P P, Lowe T M. 2009. GtRNAdb: a database of transfer RNA genes detected in genomic sequence. Nucleic Acids Res 37(Database issue): D93-97.
12. Chiang H R, Schoenfeld L W, Ruby J G, Auyeung V C, Spies N, Baek D, Johnston W K, Russ C, Luo S, Babiarz J E et al. 2010. Mammalian microRNAs: experimental evaluation of novel and previously annotated genes. Genes Dev 24(10): 992-1009.
13. Cole C, Sobala A, Lu C, Thatcher S R, Bowman A, Brown J W, Green P J, Barton G J, Hutvagner G. 2009. Filtering of deep sequencing data reveals the existence of abundant Dicer-dependent small RNAs derived from tRNAs. Rna 15(12): 2147-2160.
14. Couvillion M T, Sachidanandam R, Collins K. 2010. A growth-essential Tetrahymena Piwi protein carries tRNA fragment cargo. Genes Dev 24(24): 2742-2747.
15. Czech B, Hannon G J. 2011. Small RNA sorting: matchmaking for Argonautes. Nat Rev Genet 12(1): 19-31.
16. Czech B, Malone C D, Zhou R, Stark A, Schlingeheyde C, Dus M, Perrimon N, Kellis M, Wohlschlegel J A, Sachidanandam R et al. 2008. An endogenous small interfering RNA pathway in Drosophila. Nature 453(7196): 798-802.
17. de Lencastre A, Pincus Z, Zhou K, Kato M, Lee S S, Slack F J. 2010. MicroRNAs both promote and antagonize longevity in C. elegans. Curr Biol 20(24): 2159-2168.
18. Dhahbi J M, Atamna H, Boffelli D, Magis W, Spindler S R, Martin DIK. 2011. Deep Sequencing Reveals Novel MicroRNAs and Regulation of MicroRNA Expression during Cell Senescence. Plos One 6(5).
19. Drinnenberg I A, Fink G R, Bartel D P. 2011. Compatibility with killer explains the rise of RNAi-deficient fungi. Science 333(6049): 1592.
20. Eamens A, Wang M B, Smith N A, Waterhouse P M. 2008. RNA silencing in plants: yesterday, today, and tomorrow. Plant Physiol 147(2): 456-468.
21. Farazi T A, Horlings H M, ten Hoeve J J, Mihailovic A, Halfwerk H, Morozov P, Brown M, Hafner M, Reyal F, van Kouwenhove M et al. 2011. MicroRNA Sequence and Expression Analysis in Breast Tumors by Deep Sequencing. Cancer Res 71(13): 4443-4453.
22. Ghildiyal M, Xu J, Seitz H, Weng Z, Zamore P D. 2010. Sorting of Drosophila small silencing RNAs partitions microRNA* strands into the RNA interference pathway. Rna 16(1): 43-56.
23. Hagenbuchle 0, Larson D, Hall G I, Sprague K U. 1979. The primary transcription product of a silkworm alanine tRNA gene: identification of in vitro sites of initiation, termination and processing. Cell 18(4): 1217-1229.
24. Han J, Lee Y, Yeom K H, Kim Y K, Jin H, Kim V N. 2004. The Drosha-DGCR8 complex in primary microRNA processing. Genes Dev 18(24): 3016-3027.
25. Han J, Pedersen J S, Kwon S C, Belair C D, Kim Y K, Yeom K H, Yang W Y, Haussler D, Blelloch R, Kim V N. 2009. Posttranscriptional crossregulation between Drosha and DGCR8. Cell 136(1): 75-84.
26. Haussecker D, Huang Y, Lau A, Parameswaran P, Fire A Z, Kay M A. 2010. Human tRNA-derived small RNAs in the global regulation of RNA silencing. Rna 16(4): 673-695.
27. Jima D D, Zhang J, Jacobs C, Richards K L, Dunphy C H, Choi W W, Au W Y, Srivastava G, Czader M B, Rizzieri D A et al. 2010. Deep sequencing of the small RNA transcriptome of normal and malignant human B cells identifies hundreds of novel microRNAs. Blood 116(23): e118-127.
28. Khvorova A, Reynolds A, Jayasena S D. 2003. Functional siRNAs and miRNAs exhibit strand bias. Cell 115(2): 209-216.
29. Kim V N, Han J, Siomi M C. 2009. Biogenesis of small RNAs in animals. Nat Rev Mol Cell Biol 10(2): 126-139.
30. Koski R A, Clarkson S G. 1982. Synthesis and maturation of Xenopus laevis methionine tRNA gene transcripts in homologous cell-free extracts. J Biol Chem 257(8): 4514-4521.
31. Kozomara A, Griffiths-Jones S. 2011. miRBase: integrating microRNA annotation and deep-sequencing data. Nucleic Acids Res 39(Database issue): D152-157.
32. Kutay U, Lipowsky G, Izaurralde E, Bischoff F R, Schwarzmaier P, Hartmann E, Gorlich D. 1998. Identification of a tRNA-specific nuclear export receptor. Mol Cell 1(3): 359-369.
33. Lee R C, Feinbaum R L, Ambros V. 1993. The C. elegans heterochronic gene lin-4 encodes small RNAs with antisense complementarity to lin-14. Cell 75(5): 843-854.
34. Lee Y, Kim M, Han J, Yeom K H, Lee S, Baek S H, Kim V N. 2004a. MicroRNA genes are transcribed by RNA polymerase I I. Embo J 23(20): 4051-4060.
35. Lee Y S, Dutta A. 2009. MicroRNAs in cancer. Annu Rev Pathol 4: 199-227.
36. Lee Y S, Nakahara K, Pham J W, Kim K, He Z Y, Sontheimer E J, Carthew R W. 2004b. Distinct roles for Drosophila Dicer-1 and Dicer-2 in the siRNA/miRNA silencing pathways. Cell 117(1): 69-81.
37. Lee Y S, Shibata Y, Malhotra A, Dutta A. 2009. A novel class of small RNAs: tRNA-derived RNA fragments (tRFs). Genes Dev 23(22): 2639-2649.
38. Li Z, Ender C, Meister G, Moore P S, Chang Y, John B. 2012. Extensive terminal and asymmetric processing of small RNAs from rRNAs, snoRNAs, snRNAs, and tRNAs. Nucleic Acids Res., 40:14:6787, epub. Apr. 9, 2012.
39. Lin H F. 2007. piRNAs in the germ line. Science 316(5823): 397-397.
40. Lund E, Guttinger S, Calado A, Dahlberg J E, Kutay U. 2004. Nuclear export of microRNA precursors. Science 303(5654): 95-98.
41. Marcinowski L, Tanguy M, Krmpotic A, Radle B, Lisnic V J, Tuddenham L, Chane-Woon-Ming B, Ruzsics Z, Erhard F, Benkartek C et al. 2012. Degradation of cellular mir-27 by a novel, highly abundant viral transcript is important for efficient virus replication in vivo. PLoS Pathog 8(2): e1002510.
42. Mayr C, Bartel D P. 2009. Widespread shortening of 3′UTRs by alternative cleavage and polyadenylation activates oncogenes in cancer cells. Cell 138(4): 673-684.
43. Mi S, Cai T, Hu Y, Chen Y, Hodges E, Ni F, Wu L, Li S, Zhou H, Long C et al. 2008. Sorting of small RNAs into Arabidopsis argonaute complexes is directed by the 5′ terminal nucleotide. Cell 133(1): 116-127.
44. Nagao A, Mituyama T, Huang H, Chen D, Siomi M C, Siomi H. 2010. Biogenesis pathways of piRNAs loaded onto AGO3 in the Drosophila testis. Rna 16(12): 2503-2515.
45. Okamura K, Chung W J, Ruby J G, Guo H, Bartel D P, Lai E C. 2008. The Drosophila hairpin RNA pathway generates endogenous short interfering RNAs. Nature 453(7196): 803-806.
46. Pederson T. 2010. Regulatory RNAs derived from transfer RNA? Rna-a Publication of the Rna Society 16(10): 1865-1869.
47. Valen E, Preker P, Andersen P R, Zhao X, Chen Y, Ender C, Dueck A, Meister G, Sandelin A, Jensen T H. 2011. Biogenic mechanisms and utilization of small RNAs derived from human protein-coding genes. Nat Struct Mol Biol 18(9): 1075-1082.
48. Vaz C, Ahmad H M, Sharma P, Gupta R, Kumar L, Kulshreshtha R, Bhattacharya A. 2010. Analysis of microRNA transcriptome by deep sequencing of small RNA libraries of peripheral blood. BMC Genomics 11.
49. Wightman B, Ha I, Ruvkun G. 1993. Posttranscriptional regulation of the heterochronic gene lin-14 by lin-4 mediates temporal pattern formation in C. elegans. Cell 75(5): 855-862.
50. Xiong Y, Steitz T A. 2006. A story with a good ending: tRNA 3′-end maturation by CCA-adding enzymes. Curr Opin Struct Biol 16(1): 12-17.
51. Yekta S, Shih I H, Bartel D P. 2004. MicroRNA-directed cleavage of HOXB8 mRNA. Science 304(5670): 594-596.
52. Yi R, Qin Y, Macara I G, Cullen B R. 2003. Exportin-5 mediates the nuclear export of pre-microRNAs and short hairpin RNAs. Genes Dev 17(24): 3011-3016.
53. Zhou R, Czech B, Brennecke J, Sachidanandam R, Wohlschlegel J A, Perrimon N, Hannon G J. 2009. Processing of Drosophila endo-siRNAs depends on a specific Loquacious isoform. Rna-a Publication of the Rna Society 15(10): 1886-1895.

What is claimed is:

1. A method of diagnosing cancer, comprising measuring an amount of at least one tRF in a first biological sample from a subject and diagnosing cancer in said subject based on if a higher or lower amount of said at least one tRF is measured in the first biological sample relative to an amount of said at least one tRF in a second biological sample from a second subject without cancer or from a non-cancerous sample from said subject or in a standard.

2. The method of claim 1, wherein said at least one tRF is a tRF-1.

3. The method of claim 2, wherein said at least one tRF-1 has a sequence selected from the group having SEQ ID NOs:68-99, and homologs and fragments thereof.

4. The method of claim 3, wherein at least two tRF-1s are measured.

5. The method of claim 3, wherein each tRF-1 having a sequence selected from the group having SEQ ID NOs:68-99, and homologs and fragments thereof is measured.

6. The method of claim 1, wherein said at least one tRF is a tRF-3.

7. The method of claim 6, wherein said at least one tRF-3 has a sequence selected from the group having SEQ ID NOs:35-67, and homologs and fragments thereof.

8. The method of claim 7, wherein at least two tRF-3s are measured.

9. The method of claim 7, wherein each tRF-3 having a sequence selected from the group having SEQ ID NOs:35-67, and homologs and fragments thereof is measured.

10. The method of claim 1, wherein said at least one tRF is a tRF-5.

11. The method of claim 10, wherein said at least one tRF-5 has a sequence selected from the group having SEQ ID NOs:1-34, and homologs and fragments thereof.

12. The method of claim 11, wherein at least two tRF-5s are measured.

13. The method of claim 11, wherein each tRF-5 having a sequence selected from the group having SEQ ID NOs:1-34, and homologs and fragments thereof is measured.

14. The method of claim 1, wherein at least one tRF from at least two tRF families are measured.

15. The method of claim 14, wherein said at least two tRF families are selected from the group consisting of tRF-1, tRF-3, and tRF-5.

16. The method of claim 1, wherein at least one tRF from each at least three tRF families is measured.

17. The method of claim 1, wherein said amounts are compared on a heat map.

18. The method of claim 1, wherein said cancer is selected from the group consisting of lung cancer, B cell malignancies, squamous carcinoma of the lung, myeloid leukemia, osteosarcoma, cervical adenocarcinoma, adenocarcinoma of the colon, colon cancer, and breast cancer.

19. The method of claim 18, wherein said cancer is lung cancer.

20. The method of claim 19, wherein the amount of at least one tRF-1 is lower in said cancer.

21. The method of claim 20, wherein said tRF-1 has a sequence selected from the group consisting of SEQ ID NOs:68-99, and homologs and fragments thereof.

22. The method of claim 21, wherein the overall amount of tRF-1s measured is lower in said cancer.

23. The method of claim 18, wherein the amount of at least one tRF-3 is higher in said cancer.

24. The method of claim 23, wherein said tRF-3 has a sequence selected from the group consisting of SEQ ID NOs:35-67, and homologs and fragments thereof.

25. The method of claim 24, wherein the overall amount of tRF-3s measured is higher in said cancer.

26. The method of claim 18, wherein the amount of at least one tRF-5 is higher in said cancer.

27. The method of claim 26, wherein said tRF-5 has a sequence selected from the group consisting of SEQ ID NOs:1-34, and homologs and fragments thereof.

28. The method of claim 27, wherein the overall amount of tRF-5s measured is higher in said cancer.

29. The method of claim 28, wherein the amount of tRF-3 and the amount of tRF-5 measured are higher in said cancer and the amount of tRF-1 measured is lower in said cancer.

30. The method of claim 1, wherein said cancer is a B cell malignancy.

31. The method of claim 30, wherein said tRF is a tRF-1.

32. The method of claim 31, wherein the amount of tRF-1 measured is higher in said B cell malignancy.

33. A method for distinguishing a first cell type from a test second cell type comprising measuring an amount of at least one tRF in said first cell type and the amount of the same at least one tRF in said test second cell type, and distinguishing said first cell type from said test second cell type if a higher or lower amount of said at least one tRF is measured in said first cell type relative to an amount of said at least one tRF in said test second cell type.

34. The method of claim 33, wherein said first cell type is from an adult tissue and said test second cell type is from either an adult or an embryonic tissue.

35. The method of claim 33, wherein said first cell type is from an embryonic tissue and said test second cell type is from either an adult or an embryonic tissue.

36. The method of claim 33, wherein said method is used to distinguish the differentiation state of a cell.

37. The method of claim 36, wherein a heat map for tRF-1 is used to distinguish B-cell differentiation states of naive, plasma-cell, and germinal center cell.

38. The method of claim 36, wherein said cell is an embryonic cell.

39. The method of claim 33, wherein said cell types are from different species.

40. A tRF useful for diagnosing cancer, said tRF selected from the group consisting of tRF-1, tRF-3, and tRF-5.

41. The tRF-1 of claim 40, wherein said tRF-1 is selected from the group of tRF-1s having SEQ ID NOs:68-99, and homologs and fragments thereof.

42. The tRF-3 of claim 40, wherein said tRF-3 is selected from the group of tRF-3s having SEQ ID NOs: 35-67, and homologs and fragments thereof.

43. The tRF-5 of claim 40, wherein said tRF-5 is selected from the group of tRF-5s having SEQ ID NOs: 1-34, and homologs and fragments thereof.

44. The method of claim 1, wherein said subject is human.

45. The method of claim 1, wherein said tRF is from about 10 to about 40 nucleotide residues long.

46. The method of claim 1, wherein said tRF is detected at about 10 or more reads per million to about 10,000 or more reads per million.

47. The method of claim 46, wherein said tRF is detected at about 20 or more reads per million.

48. The method of claim 47, wherein said tRF is detected at about 100 or more reads per million.

49. The method of claim 48, wherein said tRF is detected at about 1000 or more reads per million.

50. The method of claim 49, wherein said tRF is detected at about 10,000 or more reads per million.

51. The method of claim 1, wherein said tRF amounts are higher in said cancer.

52. The method of claim 51, wherein when said tRF amounts are higher in said cancer they are at least about five times higher in said cancer.

53. The method of claim 52, wherein said tRF amounts are at least about 10 times higher in said cancer.

54. The method of claim 53, wherein said tRF amounts are at least about 50 times higher in said cancer.

55. The method of claim 54, wherein said tRF amounts are at least about 100 times higher in said cancer.

56. The method of claim 55, wherein said tRF amounts are at least about 200 times higher in said cancer.

57. The method of claim 56, wherein said tRF amounts are at least about 1000 times higher in said cancer.

58. The method of claim 1, wherein said measured amounts are compared using a heat map.

59. The method of claim 58, wherein said method distinguishes cell and tissue types for said cancer based on the z-score of the heat map.

60. A method of determining whether a tissue is normal or cancerous, said method comprising measuring an amount of at least one tRF in said tissue and determining if said tissue is normal or cancerous if a higher or lower amount of said at least one tRF is measured in said tissue relative to an amount of said at least one tRF measured in a second tissue sample known to be either normal or cancerous, thereby determining whether said tissue is normal or cancerous.

61. The method of claim 1, wherein the method confirms a previous diagnosis of cancer.

62. The method of claim 1, wherein said tRF amounts are lower in said cancer.

63. A kit for detecting and measuring tRFs in tissues and cells and for comparing amounts in normal versus cancer cells, different types of cells and of species, and for determining the differentiation state of a cell, said kit comprising at least one compound or polynucleotide of the invention, an applicator, and an instructional material for the use thereof.
